(19) United States
(12) Patent Application Publication
(10) Pub. No.: US 2026/0364169 A1
(43) Pub. Date: Nov. 19, 2026

(54) COMPOSITIONS AND METHODS OF GAMMA-DELTA T CELL EXTRACELLULAR VESICLE-BASED TUMOR VACCINES

(71) Applicant: THE UNIVERSITY OF HONG KONG, Hong Kong (CN)

(72) Inventors: Wenwei TU, Hong Kong (CN); Xiwei WANG, Hong Kong (CN); Yinping LIU, Hong Kong (CN)

(21) Appl. No.: 19/153,968

(22) PCT Filed: Feb. 18, 2024
(86) PCT No.: PCT/CN2024/077451

§ 371 Date: Aug. 6, 2025

Related U.S. Application Data
(60) Provisional application No. 63/485,734, filed on Feb. 17, 2023.

(51) Int. Cl. A61K 39/39; A61K 9/50; A61K 31/282; A61K 31/337; A61K 31/4184; A61K 31/4745; A61K 31/513; A61K 31/519; A61K 31/704; A61K 31/7048; A61K 31/7068; A61K 33/243; A61K 39/00; A61P 37/04

(52) U.S. Cl. CPC A61K 39/39; A61K 9/5068; A61K 31/282; A61K 31/337; A61K 31/4184; A61K 31/4745; A61K 31/513; A61K 31/519; A61K 31/704; A61K 31/7048; A61K 31/7068; A61K 33/243; A61K 39/001152; A61K 39/001153; A61K 39/001157; A61K 39/001168; A61K 39/001174; A61K 39/001176; A61K 39/001186; A61K 39/001188; A61K 39/001189; A61K 39/001195; A61P 37/04; A61K 2039/54; A61K 2039/543; A61K 2039/55555

(57) ABSTRACT

It has been discovered that γδ-T-extracellular vesicles (EV) have immune adjuvant effects that promote DCs maturation, mediated by the IFN-γ associated with the γδ-T-EVs. Cell-free compositions including extracellular vesicles (EVs) derived from γδ-T cells (γδ-T-EVs) and methods of use thereof are provided. The γδ-T-EVs can associated with or encapsulate tumor-associated antigens (TAAs) or peptide pools to form a γδ-T-EV vaccine composition. γδ-T-EVs compositions can be used as adjuvants in cancer immunotherapy or the γδ-T-EV vaccine compositions are to induce tumor-specific T-cell responses and direct killing of tumor cells in subjects in need thereof.

### CROSS REFERENCE TO RELATED APPLICATIONS

This application claims the benefit of and priority to U.S. Provisional Application No. 63/485,734 filed Feb. 17, 2023, the entire content of which is incorporated herein by reference for all purposes in its entirety.

### FIELD OF THE INVENTION

The disclosed invention is generally in the field of cancer vaccines and specifically in the area of personalized anti-cancer vaccines derived from gamma-delta T cells.

### BACKGROUND OF THE INVENTION

Tumor vaccines play a critical role in cancer therapy because they can amplify antigen-specific antitumor responses. Dendritic cells (DCs)-based tumor vaccines have been widely investigated, however, their clinical application is impeded due to high cost and difficulty in preparing personalized DCs. In addition, the antitumor efficacies of cell-based vaccine can be attenuated by an immunosuppressive tumor microenvironment.

Cell-free nanoparticles (NP)-based vaccines have also attracted attention for their potential in cancer therapy. NPs can protect vaccine components from degradation to improve stability, prolong bioavailability and enhance antigen uptake by antigen-presenting cells (APCs). NP size influences the uptake and specificity of NPs-based vaccine. For example, smaller NPs (20-200 nm) can be directly drained into lymph nodes and readily taken by resident DCs. Furthermore, smaller NPs are more capable of promoting DCs maturation and inducing stronger antigen-specific immunity than larger NPs. Therefore, small NPs have great potential in vaccine development. Small extracellular vesicles (EVs; 20-200 nm), such as endosome-derived exosomes, can shuttle lipids, proteins, and nucleic acids in intercellular communication, thus influencing the tumorigenesis and therapeutic outcomes of tumors. EVs can be used to develop tumor vaccines. So far, tumor-derived EVs (TEVs) and DCs-derived EVs (DC-EVs) have been mostly investigated. TEVs are rich reservoirs of tumor-associated antigens (TAAs) and can function as tumor vaccine after being loaded with immune adjuvants. Administration of TEVs-based vaccine induced broad antitumor immune responses and showed higher efficacy than tumor lysates. However, TEVs are also found to promote tumor development and metastasis, which could limit the clinical application of TEVs-based vaccine due to safety concerns. Compared to TEVs, immune cells-derived EVs have a better safety profile because of their natural immunosurveillance property. DC-EVs carried potent antigen-presenting and co-stimulatory molecules. After being loaded with immunogens, DC-EVs-based vaccine can directly induce antigen-specific immune responses or be taken by recipient APCs to indirectly promote antitumor immunity. However, the therapeutic efficacy of DC-EVs-based tumor vaccines has been unsatisfactory in clinical trials. The heterogeneity of ex vivo expanded DCs may partially account for the poor therapeutic outcomes, as EVs derived from immature DCs (iDCs) have immune-tolerant activities. In addition, the difficulties in large-scale ex vivo expansion of human DCs may also limit the preparation of DC-EVs for clinical use. Therefore, it is worth exploring other novel and more accessible EVs-based vaccines.

Human γδ-T cell is a minor T cell population that can act as innate-like T cells with direct antitumor activities against many different types of tumors. γδ-T cells also function as antigen-presenting cells (APCs) to induce antigen-specific T cell responses. More interestingly, γδ-T cells have immune adjuvant effects to induce DCs maturation and synergize CD4 T follicular helper cells to indirectly promote specific cellular and humoral immune responses. γδ-T cells derived EVs (γδ-T-EVs) displayed dual antitumor activities by carrying cytotoxic and immunostimulatory molecules, which could expand the pre-existing tumor-specific T cells. However, it remains unknown whether γδ-T-EVs can be designed as tumor vaccine to further amplify antitumor immunity.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each claim of this application.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### SUMMARY OF THE INVENTION

It has been discovered that γδ-T-extracellular vesicles (EV) have immune adjuvant effects that promote DCs maturation, mediated by the IFN-γ associated with the γδ-T-EVs. Cell-free compositions including isolated extracellular vesicles (EVs) derived from γδ-T cells (γδ-T-EVs) as the “active agents” and methods of use thereof are provided. In some forms the EVs are small EVs.

The γδ-T-EVs can be part of a heterogeneous mixture of factors such as conditioned media, or a fraction isolated therefrom. In other embodiments, γδ-T-EVs are isolated or otherwise collected from conditioned media. The γδ-T-EVs can be suspended in a pharmaceutically acceptable composition, such as a carrier or matrix or depot, prior to administration to the subject. In some embodiments the γδ-T-EVs are associated with or encapsulate tumor-associated antigens (TAAs) or peptide pools to form a γδ-T-EV vaccine composition.

Methods of using the γδ-T-EVs or γδ-T-EVs vaccine compositions to induce tumor-specific T-cell responses and direct killing of tumor cells are also described. The methods include administering the compositions of γδ-T-EVs alone (as an adjuvant) or in combination with tumor-associated antigens (TAAs) or peptide pools as a vaccine, to a subject in need thereof. In one embodiment, the γδ-T-EVs are allogenic. In another embodiment, the γδ-T-EVs are autologous. The γδ-T-EVs or γδ-T-EVs vaccine compositions are effective to increase T-cell responses in the subjects to which they are administered. Vaccine regimens are effective to induce tumor cell apoptosis. Allogeneic γδ-T-EVs-based vaccines displayed advantages over DC-EVs-based vaccines through dual antitumor activities.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings illustrate several embodiments of the disclosed method and compositions and together with the description, serve to explain the principles of the disclosed method and compositions.

FIGS. 1A-1G show that γδ-T-EVs-based vaccine efficiently induces tumor-specific T cell responses. FIG. 1A is a graph of Size distributions; FIG. 1B shows expression of EVs markers CD63 and CD81, Gray histograms represent isotype control; FIG. 1C shows expression of Alix, EBNA1 and LMP2a proteins in γδ-T-EVs and γδ-T-EVs (TAAs); FIGS. 1D-1I are graphs showing equivalent amounts of EBNA1 peptides, TAAs, γδ-T-EVs, γδ-T-EVs+free antigens, or γδ-T-EVs-based vaccines used to treat huPBMCs; after seven days, the cells were restimulated with peptide pools and subjected to detecting intracellular IFN-γ in T cells: FIGS. 1D-1E are graphs of induction of EBNA1-specific CD4 and CD8 T cells by γδ-T-EVs (EN1) and corresponding controls; FIGS. 1F-1G are graphs of induction of EBNA1 and LMP2a-specific CD4 and CD8 T cells by γδ-T-EVs (TAAs) and corresponding controls. Quantitative data are mean±SEM of four biological replicates. *p<0.05, **p<0.01, ***p<0.001. EVs: γδ-T-EVs; EN1: EBNA1; EN1a: pooled EBNA1 peptides filtrated in parallel during the preparation of γδ-T-EVs-EN1 vaccine; TAAsa: ultracentrifuged pellets from equivalent unconditioned medium containing TAAs; HIV: cells treated with γδ-T-EVs-based vaccines but restimulated with HIV p17 peptide pool.

FIGS. 2A-2N show γδ-T-EVs-based vaccine mobilizes antigen-presenting cells to induce tumor-specific T-cell responses. FIGS. 2A-2B are graphs of completed huPBMCs or HLA-DR+ cell-depleted huPBMCs treated with γδ-T-EVs-based vaccines for 7 days, then EBNA1- and LMP2a-specific T cells detected. FIG. 2C-2D Purified CD3 T cells were treated with γδ-T-EVs or γδ-T-EVs (TAAs) in the absence or presence of iDCs. TAAs treatment was also used as control (Ctr). 7 days later, LMP2a-specific T cells were detected. (FIG. 2E-2F) iDCs were treated with γδ-T-EVs or γδ-T-EVs-based vaccines for 48 h. TAAs was used as control (Ctr). The maturation makers (MHC-II, CD83, CD86 and CD40) were detected by flow cytometry. (FIG. 2G-2H) Secretion of TNF-α and IL-6 from iDCs after 48 h treatment with γδ-T-EVs or γδ-T-EVs (TAAs). (FIG. 2I-2J) iDCs were treated with γδ-T-EVs, or γδ-T-EVs (TAAs) for 48 h, then cocultured with CFSE-stained allogeneic CD3 T cells. 7 days later, proliferating T cells were determined by CFSE dilution using flow cytometry. (FIG. 2J) Secretion of IFN-γ in the coculture system of pretreated iDCs with allogeneic CD3 T cells. (FIG. 2K) Expression of IFN-γ on γδ-T-EVs or γδ-T-EVs (TAAs). (FIG. 2M-2N) iDCs were treated with γδ-T-EVs, or γδ-T-EVs (TAAs), in the presence of neutralizing anti-IFN-γ antibody or isotype control. 48 h later, the pretreated iDCs were cocultured with CFSE-stained allogeneic CD3 T cells. After 7 days, proliferating T cells were determined. Quantitative data are mean±SEM of four biological replicates. *p<0.05, **p<0.01, ***p<0.001.

FIGS. 3A-3M show that γδ-T-EVs-based vaccine preserves direct antitumor activities both in vitro and in vivo. (FIG. 3A) Apoptosis of EBV-LCL and (FIG. 3B) SNU-719 cells were determined after cultured with γδ-T-EVs or γδ-T-EVs-based vaccines for 18-24 h. Equivalent amount of PBS, EBNA1 or TAAs was used as control. (FIG. 3C) EBV-LCL cells were injected s.c. in Rag2^{-/-}γc^{-/-} mice. After 14 days, mice with subcutaneous tumors were treated with γδ-T-EVs, or γδ-T-EVs (TAAs) at indicated (n=5). TAAs was used as control (Ctr). The tumor volume (FIG. 3D) and mice survival (FIG. 3E) were determined at the indicated time. (FIG. 3F) Representative histochemical analysis of human Ki-67 in tumor tissues at the endpoints, scale bar=25 μm. (FIG. 3G) Mice with subcutaneous SNU-719 tumors were treated with γδ-T-EVs, or γδ-T-EVs (TAAs) as indicated (n=7). TAAs was used as control (Ctr). The tumor volume (FIG. 3H) and mice survival (FIG. 3I) were determined at the indicated time. (FIG. 3J) Representative histochemical analysis of human Ki-67 in tumor tissues at the endpoints, scale bar=25 μm. (FIG. 3K) Apoptosis of C666-1 NPC cells after cultured with γδ-T-EVs or γδ-T-EVs-based vaccines. (FIG. 3L-3M) Tumor volume of NPC tumor after being treated with γδ-T-EVs-based vaccines. *p<0.05, **p<0.01, ***p<0.001. ns: not significant.

FIGS. 4A-4H show γδ-T-EVs-based vaccine inhibits tumor progression in humanized mice. (FIG. 4A) Tumor models were established by injection s.c. of autologous EBV-LCL cells in humanized mice. Then, γδ-T-EVs or γδ-T-EVs-based vaccines were injected into humanized mice i.p. as indicated (n=7). TAAs was used as control (Ctr). The tumor volume (FIG. 4B) and mice survival (FIG. 4C) were determined at the indicated time. (FIG. 4D) Representative histochemical analysis of human Ki-67 in tumor tissues at the endpoints, scale bar=25 μm. (FIG. 4E-4F) γδ-T-EVs or γδ-T-EVs-based vaccines were injected into EBV-induced B cell lymphoma-bearing humanized mice biweekly for two doses. Seven days post the booster injection, the percentage of LMP2a- and EBNA1-specific T cells in peripheral blood nucleated cells were detected. (FIG. 4G) Death of EBV-LCL cells after 6 h cocultured with autologous T cells induced by γδ-T-EVs or γδ-T-EVs-based vaccines. EBV-LCL cells alone were used as control (Ctr). (FIG. 4H) Death of EBV-LCL cells with preloading of EBV peptides after 6 h cocultured with autologous T cells induced by γδ-T-EVs-based vaccines. EBV-LCL cells preloaded with HIV p17 peptides were used as control. *p<0.05, **p<0.01, ***p<0.001. ns: not significant.

FIGS. 5A-5H show allogeneic γδ-T-EVs-based vaccine also promotes antigen-presenting cells to induce tumor-specific T cell responses. Expression of MHC-II (FIG. 5A) and CD83 (FIG. 5B) on iDCs after treated with autologous, allogeneic γδ-T-EVs or γδ-T-EVs (TAAs) for 48 h. TAAs was used as control (Ctr). (FIG. 5C-5D) Secretion of IL-6 and TNF-α from iDCs after treatment. (FIG. 5E) iDCs were pretreated with autologous, allogeneic γδ-T-EVs or γδ-T-EVs (TAAs) for 48 h, then cocultured with CFSE-stained allogeneic CD3 T cells. 7 days later, proliferating T cells were determined by CFSE dilution using flow cytometry. (FIG. 5F-5G) Autologous, allogeneic γδ-T-EVs or γδ-T-EVs (TAAs) were used to treat huPBMCs. After 7 days, the cells were restimulated with EBNA1 (FIG. 5F) or LMP2a (FIG. 5G) peptide pools and subjected to detecting intracellular IFN-γ in T cells. (FIG. 5H) Completed huPBMCs or HLA-DR+ cell-depleted huPBMCs were treated with the autologous or allogeneic γδ-T-EVs (TAAs) for 7 days, then the LMP2a-specific T cells were detected. *p<0.05, **p<0.01, ***p<0.001. ns: not significant.

FIGS. 6A-6H show that allogeneic γδ-T-EVs-based vaccine inhibits tumor progression in humanized mice. FIG. 6A is a schematic showing how tumor models were established by injection s.c. of autologous EBV-LCL in humanized mice. Autologous or allogeneic γδ-T-EVs (TAAs) were injected into humanized mice i.p. at the indicated time. PBS, TAAs or γδ-T-EVs was used as control (n=8). Tumor volume FIG. 6B and FIG. 6C are graphs of mice survival, calculated as indicated. FIG. 6D. Representative histochemical analysis of human Ki-67 in tumor tissues at the endpoints, scale bar=25 μm. FIG. 6E is a schematic showing how tumor models were established by injection of autologous EBV-LCL in humanized mice reconstituted with complete huPBMCs or CD3 T cell-depleted huPBMCs. TAAs or allogeneic γδ-T-EVs (TAAs) were injected intraperitoneally into humanized mice at the indicated time (n=6). The tumor volume FIG. 6F and FIG. 6G are graphs of mice survival, calculated as indicated. FIG. 6H. Representative histochemical analysis of human Ki-67 in tumor tissues at the endpoints, scale bar=25 μm. *p<0.05, **p<0.01, ***p<0.001. ns: not significant; Auto-: autologous; Allo-: allogeneic.

FIGS. 7A-7E show that allogeneic γδ-T-EVs-based vaccine prevents tumor development in humanized mice. FIG. 7A is a schematic showing how humanized mice were primed and boosted using autologous or allogeneic γδ-T-EVs-based vaccine as indicated, then EGFP-expressing EBV-LCL cells were subcutaneously injected one week later (n=8). Equivalent amount of PBS, TAAs or γδ-T-EVs was used as control. FIG. 7B is a graph of Tumor incidence; FIG. 7C. whole-body fluorescence images of mice 42 days after tumor cell inoculation. FIG. 7D is a graph of tumor volume, and FIG. 7E is a graph of mice survival, determined as indicated. ***p<0.001. ns: not significant; Auto-: autologous; Allo-: allogeneic.

FIGS. 8A-8J show that allogeneic γδ-T-EVs-based vaccine has advantages over DC-EVs-based vaccine by displaying dual antitumor activities. FIGS. 8A-8B are graphs of secretion of TNF-α and IL-6 from iDCs after 48 h treatment with allogeneic γδ-T-EVs, DC-EVs, γδ-T-EVs (TAAs) or DC-EVs (TAAs). TAAs was used as control (Ctr). FIG. 8C is a graph of iDCs treated with allogeneic γδ-T-EVs, DC-EVs, Y&-T-EVs (TAAs) or DC-EVs (TAAs) for 48 h, TAAs was used as control (Ctr). Then, the pretreated-iDCs were cocultured with CFSE-stained allogeneic CD3 T cells. 7 days later, proliferating T cells were determined by CFSE dilution using flow cytometry. FIG. 8D is a graph showing secretion of IFN-γ in the coculture system of pretreated iDCs with allogeneic CD3 T cells. FIG. 8E is a graph of allogeneic γδ-T-EVs- or DC-EVs-based vaccine used to treat huPBMCs. After 7 days, the cells were restimulated with LMP2a peptide pools and subjected to detecting intracellular IFN-γ in T cells. FIG. 8F is a graph of apoptosis of EBV-LCL cells after being cultured with allogeneic γδ-T-EVs- or DC-EVs-based vaccine for 18-24 h. FIG. 8G is a schematic showing how tumor models were established by injection s.c. of EBV-LCL cells in humanized mice. Then, allogeneic γδ-T-EVs-based or DC-EVs-based vaccine were injected into humanized mice i.p. at the indicated time (n=6 or 7). TAAs was used as control (Ctr). FIGS. 8H-8I are graphs showing tumor volume (FIG. 8H) and mice survival (FIG. 8I), calculated as indicated. FIG. 8J. Representative histochemical analysis of human Ki-67 in tumor tissues at the endpoints. scale *p<0.05, **p<0.01, ***p<0.001. ns: not significant.

FIG. 9A is a graph of size distribution of DC-EVs- and γδ-T-EVs-based vaccines. Size distribution of γδ-T-EVs, DC-EVs, γδ-T-EVs (TAAs) or DC-EVs (TAAs) determined by Zeta View Particle Tracking Analyzer. Data was shown from one of three independent experiments. FIG. 9B shows expression of IFN-γ on DC-EVs- and γδ-T-EVs-based vaccines. Expression of IFN-γ on γδ-T-EVs, DC-EVs, γδ-T-EVs (TAAs) or DC-EVs (TAAs) was determined by flow cytometry. Gray histograms represent isotype control. Data was shown from one of three independent experiments.

FIG. 10A-10D shows apoptosis of multiple tumor cells after treated with γδ-T-EVs-based vaccines or PBS (Ctr).

FIG. 11A-11C show induction of antigen-specific T cells from human PBMCs after 7 days by γδ-T-EVs-based vaccines prepared via pulsed with TAAs (melanoma, cervical cancer) or recombinant tumor antigen (liver cancer). The TAAs are cellular lysates obtained after freeze-thaw treatment of respective cancer cell lines ((e.g., EBV-associated B-cell lymphoma, melanoma, cervical and liver cancer).

### DETAILED DESCRIPTION OF THE INVENTION

The disclosed method and compositions may be understood more readily by reference to the following detailed description of particular embodiments and the Example included therein and to the Figures and their previous and following

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compositions may not be explicitly disclosed, each is specifically contemplated and described herein. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. Further, each of the materials, compositions, components, etc. contemplated and disclosed as above can also be specifically and independently included or excluded from any group, subgroup, list, set, etc. of such materials. These concepts apply to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

### I. Definitions

As used herein, the term “carrier” or “excipient” refers to an organic or inorganic ingredient, natural or synthetic inactive ingredient in a formulation, with which one or more active ingredients are combined.

The term “preventing” as used herein refers to administering a composition prior to the onset of clinical symptoms of a disease or conditions so as to prevent a physical manifestation of aberrations associated with the disease or condition.

The term “in need of treatment” as used herein refers to a judgment made by a caregiver (e.g. physician, nurse, nurse practitioner, or individual in the case of humans; veterinarian in the case of animals, including non-human mammals) that a subject requires or will benefit from treatment. This judgment is made based on a variety of factors that are in the realm of a care giver's expertise, but that include the knowledge that the subject is ill, or will be ill, as the result of a condition that is treatable by the compositions of the invention.

As used herein, “subject” includes, but is not limited to, animals, plants, bacteria, viruses, parasites and any other organism or entity. The subject can be a vertebrate, more specifically a mammal (e.g., a human, horse, pig, rabbit, dog, sheep, goat, non-human primate, cow, cat, guinea pig or rodent), a fish, a bird or a reptile or an amphibian. The subject can be an invertebrate, more specifically an arthropod (e.g., insects and crustaceans). The term does not denote a particular age or sex. Thus, adult and newborn subjects, as well as fetuses, whether male or female, are intended to be covered. A patient refers to a subject afflicted with a disease or disorder. The term “patient” includes human and veterinary subjects.

By “treatment” and “treating” is meant the medical management of a subject with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. It is understood that treatment, while intended to cure, ameliorate, stabilize, or prevent a disease, pathological condition, or disorder, need not actually result in the cure, amelioration, stabilization or prevention. The effects of treatment can be measured or assessed as described herein and as known in the art as is suitable for the disease, pathological condition, or disorder involved. Such measurements and assessments can be made in qualitative and/or quantitative terms. Thus, for example, characteristics or features of a disease, pathological condition, or disorder and/or symptoms of a disease, pathological condition, or disorder can be reduced to any effect or to any amount.

By the term “effective amount” of a composition as provided herein is meant a nontoxic but sufficient amount of the composition to provide the desired result. As will be pointed out below, the exact amount required will vary from subject to subject, depending on the species, age, and general condition of the subject, the severity of the disease that is being treated, the particular composition/agent used, its mode of administration, and the like. Thus, it is not possible to specify an exact “effective amount.” However, an appropriate effective amount can be determined by one of ordinary skill in the art using only routine experimentation.

By “pharmaceutically acceptable” is meant a material that is not biologically or otherwise undesirable, i.e., the material can be administered to a subject along with the selected composition/agent without causing any undesirable biological effects or interacting in a deleterious manner with any of the other components of the pharmaceutical composition in which it is contained.

As used herein, the term “pharmaceutically acceptable carrier” encompasses any of the standard pharmaceutical carriers, such as a phosphate buffered saline solution, water and emulsions such as an oil/water or water/oil emulsion, and various types of wetting agents.

As used herein, the term “purified,” “isolated,” and like terms relate to the isolation of a molecule/active agent(s) in a form that is substantially free (at least 60% free, preferably 75% free, and most preferably 90% free) from other components normally associated with the molecule/active agents in a native environment.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%; in other forms the values may range in value either above or below the stated value in a range of approx. +/−5%; in other forms the values may range in value either above or below the stated value in a range of approx. +/−2%; in other forms the values may range in value either above or below the stated value in a range of approx. +/−1%. The preceding ranges are intended to be made clear by context, and no further limitation is implied.

### II. Compositions

### A. γδ-T-EVs Compositions

Cell-free compositions including extracellular vesicles (EVs) derived from γδ-T cells (γδ-T-EVs), alone or in combination with tumor antigens, are provided.

The γδ-T-EVs can be part of a heterogeneous mixture of factors such as conditioned media, or a fraction isolated therefrom. In other embodiments, γδ-T-EVs, or one or more subtypes thereof, are isolated or otherwise collected from conditioned media. The γδ-T-EVs, or one or more subtypes thereof, can be suspended in a pharmaceutically acceptable composition, such as a carrier or matrix or depot, prior to administration to the subject. In some embodiments the γδ-T-EVs are associated with or encapsulate tumor-associated antigens (TAAs) or peptide pools. In some forms the TAA's are provided by cellular lysates obtained after freeze-thaw treatment of respective a cancer cell line. For example, the TAAs can be provided by obtaining tumor lysates of EBV-LCL cells, via freeze-thaw methods known in the art (Hatfield, et al., J Immunother. 2008, 31 (7): 620-32). The TAAs can be provided by obtaining tumor lysates of cancer cells via freeze-thaw, such as colorectal carcinoma, RCC (renal cell carcinoma), prostate carcinoma, cervical carcinoma, melanoma, lung carcinoma, breast carcinoma, B, T leukemia, lymphoma, pancreatic carcinoma, myeloma, nasopharyngeal carcinoma, gastric cancer cells, etc. In some forms, the TAAs are provided by lysates isolated from a patient's own tumor as the TAA source. However, TAAs can be provided by lysates isolated from a source other than the patient's own tumor, as the TAA source. Whole-tumor lysate (WTL) constitutes a valid alternative source of cancer antigens that circumvents the need for antigen identification and validation.

Compositions containing γδ-T-EVs are provided in effective amounts to function as an adjuvant, measured for example, by their ability to promote the maturation of dendritic cells (DCs), as revealed by enhanced expression of maturation markers, secretion of pro-inflammatory cytokines and antigen-presenting ability of DCs after γδ-T-EVs administration, measured for example as upregulation of CD83 and MHC-II expression compared with TAAs treatment by at least 30, at least 40, or at least 50% compared to no treatment with γδ-T-EVs.

Extracellular vesicles (EVs) are a heterogenous group of membrane-surrounded structures. EVs, broadly comprised of microvesicles and exosomes, are released to the extracellular environment from nearly all cells either via shedding from the plasma membrane or by originating from the endosomal system. Exosomes are 40-150 nm, endosome-derived small EVs (sEVs) that are released by cells into the extracellular environment. In 2018, the International Society on Extracellular Vesicles (ISEV) endorsed EV as the generic term to be used for particles of any cellular origin that lack a nucleus and are delimited by a lipid bilayer. One class of characterization parameters include: (1) Size of EVs-small EVs (100-200 nm), in some forms from 20-200 nm, large EVs (200-1,000 nm); (2) Sedimentation or density of EVs-low, middle, or high; (3) Marker expression—e.g., CD63, CD81, or Annexin A1-expressing EVs; (4) Types of cells—e.g., EVs-derived from γδ-T cells; and (5) Biogenesis—e.g., plasma membrane or endosome. (Reviewed in Li, et al., Front in Immunol., vol. 11, article 1525 (2020). Vs harbor a cargo consisting of proteins, mRNA, non-coding (nc) RNAs such as microRNAs and long nc-RNAs, DNA, and lipids. This cargo can be transferred to recipient cells, which constitutes an important form of physiological cell-to-cell communication. The disclosed EVs express the markers CD 63 and CD 81.

The γδ-T-EVs can be associated with, or encapsulate one or more tumor antigens. Tumor antigens as used herein include tumor-associated antigens (TAAs) (are relatively restricted to tumor cells) and tumor-specific antigens (TSAs) (are unique to tumor cells). γδ-T-EVs are directly loaded with tumor antigen EBNA1 (γδ-T-EVs (EN1)) or indirectly loaded with TAAs (γδ-T-EVs (TAAs)) after pulsing, before EV isolation as demonstrated in the Example section of this application. In some forms, the γδ-T-EVs are directly loaded with tumor antigen EBNA1 (γδ-T-EVs (EN1)) or indirectly loaded with TAAs (γδ-T-EVs (TAAs)) after pulsing, before EV isolation. “Pulsing” as used herein means: γδ-T cells were treated/exposed with TAAs at the indicated time before EVs isolation. As used herein, “direct loading” involves the loading of antigens onto EVs through physical or chemical methods after isolating the EVs from cell culture supernatant. As used herein, “indirect loading” entails feeding the cells with antigens. These antigens will be engulfed by γδ-T cells and subsequently encapsulated into EVs. The antigen-containing EVs are then secreted into the cell culture supernatant, which can be subjected to EVs isolation.

In some forms the TAA's are provided by cellular lysates obtained after freeze-thaw treatment of respective a cancer cell line.

Tumor antigens are proteins expressed on the surface of tumor cells or secreted into the bloodstream. Tumor antigens have been identified for most human cancers. There are two major classes of tumor antigens. Tumor-specific antigens (TSA), are found only on cancer cells, not on healthy cells and tumor-associated antigens (TAA), which are highly expressed on tumor cells, but are also expressed at lower levels on healthy cells. Examples of tumor antigens include, but are not limited to EBV antigens such as Epstein-Barr nuclear antigen 1 (EBNA1) and latent membrane protein 2a (LMP2a) is shown in Table 12-1 in. Zarour H M, DeLeo A, Finn O J, et al. Categories of Tumor Antigens. In: Kufe D W, Pollock R E, Weichselbaum R R, et al., editors. Holland-Frei Cancer Medicine. 6th edition. Hamilton (ON): BC Decker; 2003. Available from: https://www.ncbi. nlm. nih. gov/books/NBK12961/

TABLE 1
General Categories and Examples of Tumor Antigens

| Category | Example Antigen | Cancer Histology |
|---|---|---|
| Oncofetal | CEA (carcinoembryonic | Colorectal carcinoma |
| | antigen) | |
| | Immature laminin receptor | RCC |
| | TAG-72 | Prostate carcinoma |
| Oncoviral | HPV E6, E7 | Cervical carcinoma |
| Overexpressed/ | BING-4 | Melanoma |
| accumulated | | |
| | Calcium-activated chloride | Lung carcinoma |
| | channel 2 | |
| | Cyclin-B₁ | Multi |
| | 9D7 | RCC |
| | Ep-CAM | Breast carcinoma |
| | EphA3 | Multi |
| | Her2/neu | Multi |
| | Telomerase | Multi |
| | Mesothelin | Ductal pancreatic |
| | | carcinoma |
| | SAP-1 | Colorectal carcinoma |
| | Survivin | Multi |
| Cancer-Testis | BAGE family | Multi |
| | CAGE family | Multi |
| | GAGE family | Multi |
| | MAGE family | Multi |
| | SAGE family | Multi |
| | XAGE family | Multi |
| CT9, CT10 | | Multi |
| | NY-ESO-1/LAGE-1 | Multi |
| | PRAME | Multi |
| | SSX-2 | Melanoma, Multi |
| Lineage | Melan-A/MART-1 | Melanoma |
| Restricted | | |
| | Gp100/pmel17 | Melanoma |
| | Tyrosinase | Melanoma |
| | TRP-1/-2 | Melanoma |
| | P.polypeptide | Melanoma |
| | MC1R | Melanoma |
| | Prostate-specific antigen | Prostate |
| Mutated | β-catenin | Melanoma, Prostate, |
| | | HCC |
| | BRCA1/2 | Breast, ovarian |
| | | carcinoma |
| | CDK4 | Multi |
| | CML66 | CML |
| | Fibronectin | Multi |
| | MART-2 | Melanoma |
| | p53 | Multi |
| | Ras | Multi ® |
| | TGF-βRII | Colorectal carcinoma |
| Posttranslationally | MUC1 (Mucin short | Ductal carcinoma, |
| altered | variant S1) | RCC. |
| Idiotypic | Ig, TCR | B. T leukemia, |
| | | lymphoma, myeloma |

BRCA = breast cancer antigen; CDK4 = cyclin-dependent kinase-4; CEA = carcino-embryonic antigen; CML66 = chronic myelogenous leukemia (antigen) 66; CT = cancer testis; HPV = human papilloma virus; Ep-CAM = epithelial cell adhesion molecule; Ig = immunoglobulin; MART-1/-2 = melanoma antigen recognized by T cells-1/-2; MC1R = melanocortin-1-receptor; SAP-1 = stomach cancer-associated protein tyrosine phosphatase-1; TAG-72 = tumor antigen-72; TCR = T cell receptor; TGF-βRII = transforming growth factor-β receptor II; TRP = tyrosinase-related protein.

Other examples of tumor antigens include Alphafetoprotein (AFP), Epithelial tumor antigen (ETA) and Mucin-16 (MUC-16) also known as Ovarian cancer-related tumor marker CA125 (Cancer Antigen 125); MAGE (Melanoma-Associated Antigen); Glypican-3 (GPC3); SOX2; WT1 (Wilms Tumor 1); HMGA2 (High Mobility Group AT-Hook 2); HSP90 (Heat Shock Protein 90); PSMA (Prostate-Specific Membrane Antigen); CA19-9 (Carbohydrate Antigen 19-9) and MUC16 (CA125). Identification and characterization of 9D7 is disclosed in Klade, et al., doi.org/10.1002/ijc. 1582.

In some forms, the γδ-T-EVs is not associated with, or does not encapsulate a microRNA such as miR 138.

### B. Pharmaceutical Compositions

Pharmaceutical compositions including EVs are also provided. Pharmaceutical compositions can be administered parenterally (intramuscular (IM), intraperitoneal (IP), intravenous (IV), subcutaneous injection (SubQ), subdermal), transdermally (either passively or using iontophoresis or electroporation), or by any other suitable means, and can be formulated in dosage forms appropriate for each route of administration.

The vesicles described herein can be conveniently formulated into pharmaceutical compositions in association with a pharmaceutically acceptable carrier. See, e.g., Remington's Pharmaceutical Sciences, latest edition, by E. W. Martin Mack Pub. Co., Easton, PA, which discloses typical carriers and conventional methods of preparing pharmaceutical compositions that can be used in conjunction with the preparation of formulations of the active agents (i.e., γδ-T-extracellular vesicles) described herein. These most typically would be standard carriers for administration of compositions to humans. In one aspect, humans and non-humans, including solutions such as sterile water, saline, and buffered solutions at physiological pH. Other compounds will be administered according to standard procedures used by those skilled in the art.

Preparations for parenteral administration include sterile aqueous or non-aqueous solutions, suspensions, and emulsions which can also contain buffers, diluents and other suitable additives. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives can also be present such as, for example, antimicrobials, anti-oxidants, chelating agents, and inert gases and the like.

For example, parenteral administration may include administration to a patient intravenously, intradermally, intraarterially, intraperitoneally, intralesionally, intracranially, intraarticularly, intraprostatically, intrapleurally, intratracheally, intravitreally, intratumorally, intramuscularly, subcutaneously, subconjunctivally, intravesicularly, intrapericardially, intraumbilically, by injection, and by infusion.

Parenteral formulations can be prepared as aqueous compositions using techniques known in the art. Typically, such compositions can be prepared as injectable formulations, for example, solutions or suspensions; solid forms suitable for using to prepare solutions or suspensions upon the addition of a reconstitution medium prior to injection; emulsions, such as water-in-oil (w/o) emulsions, oil-in-water (o/w) emulsions, and microemulsions thereof, liposomes, or emulsomes.

The carrier can be a solvent or dispersion medium containing, for example, water, ethanol, one or more polyols (e.g., glycerol, propylene glycol, and liquid polyethylene glycol), oils, such as vegetable oils (e.g., peanut oil, corn oil, sesame oil, etc.), and combinations thereof. The proper fluidity can be maintained, for example, by the use of a coating, such as lecithin, by the maintenance of the required particle size in the case of dispersion and/or by the use of surfactants. In many cases, it will be preferable to include isotonic agents, for example, sugars or sodium chloride.

Solutions and dispersions of the active agent disclosed herein (γδ-T-extracellular vesicles) as the free acid or base or pharmacologically acceptable salts thereof can be prepared in water or another solvent or dispersing medium suitably mixed with one or more pharmaceutically acceptable excipients including, but not limited to, surfactants, dispersants, emulsifiers, pH modifying agents, viscosity modifying agents, and combination thereof.

Suitable surfactants may be anionic, cationic, amphoteric or nonionic surface-active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-.beta.-alanine, sodium N-lauryl-.beta.-iminodipropionate, myristoamphoacetate, lauryl betaine and lauryl sulfobetaine.

The formulation can contain a preservative to prevent the growth of microorganisms. Suitable preservatives include, but are not limited to, parabens, chlorobutanol, phenol, sorbic acid, and thimerosal. The formulation may also contain an antioxidant to prevent degradation of the active agent(s).

The formulation is typically buffered to a pH of 3-8 for parenteral administration upon reconstitution. Suitable buffers include, but are not limited to, phosphate buffers, acetate buffers, and citrate buffers.

Water-soluble polymers are often used in formulations for parenteral administration. Suitable water-soluble polymers include, but are not limited to, polyvinylpyrrolidone, dextran, carboxymethylcellulose, and polyethylene glycol.

Sterile injectable solutions can be prepared by incorporating the active agent in the required amount in the appropriate solvent or dispersion medium with one or more of the excipients listed above, as required, followed by filtered sterilization. Generally, dispersions are prepared by incorporating the various sterilized active ingredients into a sterile vehicle which contains the basic dispersion medium and the required other ingredients from those listed above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. The powders can be prepared in such a manner that the particles are porous in nature, which can increase dissolution of the particles. Methods for making porous particles are well known in the art. The parenteral formulations described herein can be formulated for controlled release including immediate release, delayed release, extended release, pulsatile release, and combinations thereof.

For parenteral administration, the active agents, and optional one or more additional active agents, can be incorporated into microparticles, nanoparticles, or combinations thereof that provide controlled release of the active agents and/or one or more additional active agents. In embodiments wherein the formulations contains two or more drugs, the drugs can be formulated for the same type of controlled release (e.g., delayed, extended, immediate, or pulsatile) or the drugs can be independently formulated for different types of release (e.g., immediate and delayed, immediate and extended, delayed and extended, delayed and pulsatile, etc.).

Formulations for topical administration can include ointments, lotions, creams, gels, drops, suppositories, sprays, liquids and powders. Conventional pharmaceutical carriers, aqueous, powder or oily bases, thickeners and the like can be necessary or desirable.

Compositions for oral administration can include powders or granules, suspensions or solutions in water or non-aqueous media, capsules, sachets, or tablets. Suitable oral dosage forms include tablets, capsules, solutions, suspensions, syrups, and lozenges. Tablets can be made using compression or molding techniques well known in the art. Gelatin or non-gelatin capsules can prepared as hard or soft capsule shells, which can encapsulate liquid, solid, and semi-solid fill materials, using techniques well known in the art. Thickeners, flavorings, diluents, emulsifiers, dispersing aids or binders can be desirable.

Formulations may be prepared using a pharmaceutically acceptable carrier. As generally used herein “carrier” includes, but is not limited to, diluents, preservatives, binders, lubricants, disintegrators, swelling agents, fillers, stabilizers, and combinations thereof.

Carrier also includes all components of the coating composition, which may include plasticizers, pigments, colorants, stabilizing agents, and glidants.

Examples of suitable coating materials include, but are not limited to, cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Oral dosage forms, such as capsules, tablets, solutions, and suspensions, can for formulated for controlled release. For example, the active agents (γδ-T-extracellular vesicles) and optional one or more additional active agents can be formulated into nanoparticles, microparticles, and combinations thereof, and encapsulated in a soft or hard gelatin or non-gelatin capsule or dispersed in a dispersing medium to form an oral suspension or syrup. The particles can be formed of the drug and a controlled release polymer or matrix. Alternatively, the drug particles can be coated with one or more controlled release coatings prior to incorporation in to the finished dosage form.

The formulation can be in the form of a suspension or emulsion. In general, pharmaceutical compositions are provided including effective amounts of one or more active agents optionally include pharmaceutically acceptable diluents, preservatives, solubilizers, emulsifiers, adjuvants and/or carriers. Such compositions can include diluents, sterile water, buffered saline of various buffer content (e.g., Tris-HCl, acetate, phosphate) at various pHs and ionic strengths; and optionally, additives such as detergents and solubilizing agents (e.g., TWEEN® 20, TWEEN® 80 also referred to as polysorbate 20 or 80), anti-oxidants (e.g., ascorbic acid, sodium metabisulfite), and preservatives (e.g., Thimersol, benzyl alcohol) and bulking substances (e.g., lactose, mannitol). Examples of non-aqueous solvents or vehicles are propylene glycol, polyethylene glycol, vegetable oils, such as olive oil and corn oil, gelatin, and injectable organic esters such as ethyl oleate. The formulations may be lyophilized and redissolved/resuspended immediately before use. The formulation may be sterilized by, for example, filtration through a bacterium retaining filter, by incorporating sterilizing agents into the compositions, by irradiating the compositions, or by heating the compositions.

“Diluents”, also referred to as “fillers,” are typically necessary to increase the bulk of a solid dosage form so that a practical size is provided for compression of tablets or formation of beads and granules. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powdered sugar.

“Binders” are used to impart cohesive qualities to a solid dosage formulation, and thus ensure that a tablet or bead or granule remains intact after the formation of the dosage forms. Suitable binder materials include, but are not limited to, starch, pregelatinized starch, gelatin, sugars (including sucrose, glucose, dextrose, lactose and sorbitol), polyethylene glycol, waxes, natural and synthetic gums such as acacia, tragacanth, sodium alginate, cellulose, including hydroxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone.

“Lubricants” are used to facilitate tablet manufacture. Examples of suitable lubricants include, but are not limited to, magnesium stearate, calcium stearate, stearic acid, glycerol behenate, polyethylene glycol, talc, and mineral oil.

“Disintegrants” are used to facilitate dosage form disintegration or “breakup” after administration, and generally include, but are not limited to, starch, sodium starch glycolate, sodium carboxymethyl starch, sodium carboxymethylcellulose, hydroxypropyl cellulose, pregelatinized starch, clays, cellulose, alginine, gums or cross linked polymers, such as cross-linked PVP (Polyplasdone®XL from GAF Chemical Corp).

“Stabilizers” are used to inhibit or retard drug decomposition reactions, which include, by way of example, oxidative reactions. Suitable stabilizers include, but are not limited to, antioxidants, butylated hydroxytoluene (BHT); ascorbic acid, its salts and esters; Vitamin E, tocopherol and its salts; sulfites such as sodium metabisulphite; cysteine and its derivatives; citric acid; propyl gallate, and butylated hydroxyanisole (BHA).

Immunologic adjuvants stimulate the immune system's response to a target antigen, but do not provide immunity themselves. Adjuvants can act in various ways in presenting an antigen to the immune system. Adjuvants can act as a depot for the antigen, presenting the antigen over a longer period of time, thus maximizing the immune response before the body clears the antigen. Examples of depot type adjuvants are oil emulsions. An adjuvant can also act as an irritant, which engages and amplifies the body's immune response.

The adjuvant may be without limitation alum (e.g., aluminum hydroxide, aluminum phosphate); saponins purified from the bark of the Q. saponaria tree such as Quil A (a mixture of more than 25 different saponin molecules), or subcombinations or individual molecules thereof such as QS21 (aglycolipid that elutes in the 21st peak with HPLC fractionation; Antigenics, Inc., Worcester, Mass.); poly [di(carboxylatophenoxy) phosphazene (PCPP polymer; Virus Research Institute, USA), Flt3 ligand, Leishmania elongation factor (apurified Leishmania protein; Corixa Corporation, Seattle, Wash.), ISCOMS (immunostimulating complexes which contain mixed saponins, lipids and form virus-sized particles with pores that can hold antigen; CSL, Melbourne, Australia), Pam3Cys, SB-AS4 (SmithKline Beecham adjuvant system #4 which contains alum and MPL; SBB, Belgium), non-ionic block copolymers that form micelles such as CRL 1005 (these contain a linear chain of hydrophobic polyoxypropylene flanked by chains of polyoxyethylene, Vaxcel, Inc., Norcross, Ga.), and Montanide IMS (e.g., IMS 1312, water-based nanoparticles combined with a soluble immunostimulant, Seppic).

Adjuvants may be TLR ligands, such as those discussed above. Adjuvants that act through TLR3 include without limitation double-stranded RNA. Adjuvants that act through TLR4 include without limitation derivatives of lipopolysaccharides such as monophosphoryl lipid A (MPLA; Ribi ImmunoChem Research, Inc., Hamilton, Mont.) and muramyl dipeptide (MDP; Ribi) andthreonyl-muramyl dipeptide (t-MDP; Ribi); OM-174 (a glucosamine disaccharide related to lipid A; OM Pharma SA, Meyrin, Switzerland). Adjuvants that act through TLR5 include without limitation flagellin. Adjuvants that act through TLR7 and/or TLR8 include single-stranded RNA, oligoribonucleotides (ORN), synthetic low molecular weight compounds such as imidazoquinolinamines (e.g., imiquimod (R-837), resiquimod (R-848)). Adjuvants acting through TLR9 include DNA of viral or bacterial origin, or synthetic oligodeoxynucleotides (ODN), such as CpG ODN. Another adjuvant class is phosphorothioate containing molecules such as phosphorothioate nucleotide analogs and nucleic acids containing phosphorothioate backbone linkages.

The adjuvant can also be oil emulsions (e.g., Freund's adjuvant); saponin formulations; virosomes and viral-like particles; bacterial and microbial derivatives; immunostimulatory oligonucleotides; ADP-ribosylating toxins and detoxified derivatives; alum; BCG; mineral-containing compositions (e.g., mineral salts, such as aluminium salts and calcium salts, hydroxides, phosphates, sulfates, etc.); bioadhesives and/or mucoadhesives; microparticles; liposomes; polyoxyethylene ether and polyoxyethylene ester formulations; polyphosphazene; muramyl peptides; imidazoquinolone compounds; and surface active substances (e.g. lysolecithin, pluronic polyols, polyanions, peptides, oil emulsions, keyhole limpet hemocyanin, and dinitrophenol).

Adjuvants may also include immunomodulators such as cytokines, interleukins (e.g., IL-1, IL-2, IL-4, IL-5, IL-6, IL-7, IL-12, etc.), interferons (e.g., interferon-. gamma.), macrophage colony stimulating factor, and tumor necrosis factor.

Immunostimulatory complexes called ISCOMs are particulate antigen delivery systems having antigen, cholesterol, phospholipid and saponin (Quil A or other saponin) with potent immunostimulatory activity. ISCOMATRIX® is a particulate adjuvant having cholesterol, phospholipids and saponins (Quil A) but without containing antigen. See, e.g., U.S. Pat. No. 9,149,520. This adjuvant has principally the same structure as ISCOMs, consisting of perforated cage-like particles of approximately 40 nm in diameter. The antigens can be formulated with ISCOMATRIX® to produce vaccines capable of antigen presentation and immunostimulants similar to ISCOMs-type formulations, but with a wider range of applicability, since its use is not limited to hydrophobic membrane proteins. Modifications of ISCOMs formulations and ISCOMATRIX® have also been developed to achieve a better association of some antigens, such as described in WO 98/36772.

ISCOMs and ISCOMATRIX® combine the advantages of a particulate delivery system with the in situ presence of an adjuvant (Quil A) and consequently have been found to be more immunogenic than other colloidal systems such as liposomes and protein micelles. Formulations of ISCOMs and ISCOMATRIX® retained the adjuvant activity of the Quil A, while increasing its stability, reducing its hemolytic activity, and producing less toxicity. They also generate a similar immune response to the one obtained by immunizing with simple mixtures of antigen and saponin, but allow for the use of substantially smaller amounts of antigen. Several ISCOMs-type vaccine formulations or containing ISCOMATRIX® have been approved for veterinary use, for example against equine influenza virus.

Other liposomal systems mainly composed of saponins from Q. saponaria and sterols (primarily cholesterol) have been described, one of which is referred to as ASO1B. See, e.g., WO 96/33739, being also formulated as emulsions such as described in US 2005/0220814. See, also, U.S. Published Application No. 2011/0206758.

Iscomatrix-like adjuvants such as ISCOMATRIX® are thought to function via canonical inflammasome activation and subsequent release of pro-inflammatory cytokines such as IL-18 and IL-1ß (Wilson, et al., Journal of Immunology. 2014; 192 (7): 3259-68. doi: 10.4049/jimmunol. 1302011. PubMed PMID: 24610009). This mechanism is thought to be mediated at least in-part by endosomal degradation and the release of NRLP3-activating cathepsin proteases into the cytosol.

Transdermal formulations may also be prepared. These will typically be ointments, lotions, sprays, or patches, all of which can be prepared using standard technology. Transdermal formulations can include penetration enhancers. Chemical enhancers and physical methods including electroporation and microneedles can work in conjunction with this method. Typically the penetration enhancer(s) are selected such that it/they do not disrupt and/or eliminate the biological activity of the EVs.

### C. Methods of Making γδ-T-EVs

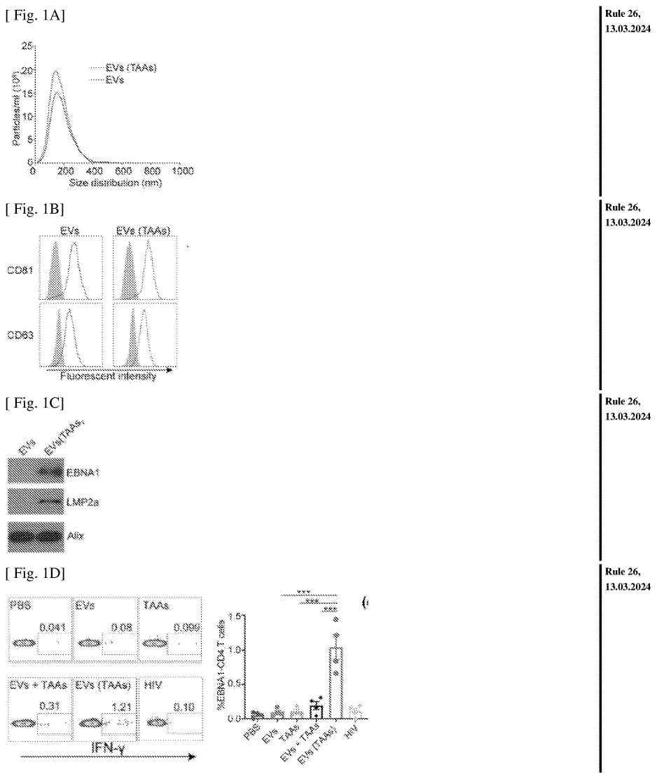

Methods for isolating EVs derived from γδ-T cells (γδ-T-EVs) are described in Wang, X., et al., Sci Transl Med, 2020.12: p. eaaz3426; Wang, X., et al., J Immunother Cancer, 2022.10 (2): e003832 (2022) and CN 108103026. The improved methods herein improve yield over the methods in CN 108103026, whose protocol includes expanding γδ-T cells using 5 μM zoledronate and 1000 IU/ml IL-2, which only yielded around 60% purity of γδ-T cells after expansion. Prior to preparing γδ-T-EVs, the γδ-T cells needed to be sorted to achieve high purity (CN 108103026, FIG. 1B). However, cell sorting procedures are time-consuming and can reduce cell viability, potentially impacting the production and function of γδ-T-EVs. By contrast, the improved protocol disclosed herein uses pamidronate (about 9 μg/ml) IL-2 (about 200 IU/ml IL-2), and included restimulating the γδ-T cells three days post-initial activation; this results in high purity (>95%) of γδ-T cells. This expansion protocol allows for to directly subjecting the expanded γδ-T cells to the preparation of γδ-T-EVs without the need for prior cell sorting. This approach is considerably more convenient in practice and better preserves the viability of γδ-T cells and the function of γδ-T-EVs.

The γδ-T-EVs are associated with or encapsulate tumor-associated antigens (TAAs) (herein, γδ-T-EVs (TAAs) or peptide pools, to form a γδ-T-EV vaccine composition. CN 108103026, discloses γδ-T-EVs carrying miR-138, whereby γδ-T cells transfected with a lentivirus (miR-138) vector. However, due to the innate-like nature of γδ-T cells with intrinsic antivirus abilities, transfection with exogenous virus can be challenging and may reduce cell viability of γδ-T cells. Moreover, the use of vector derived from retrovirus in γδ-T-EVs preparation could introduce additional bioactive hazards. Additionally, only a single antitumor agent (miR-138) was loaded into their γδ-T-EVs product using the lentivirus vector.

The disclosed methods prepare γδ-T-EVs (TAAs) by directly exposing γδ-T cells to TAAs. Leveraging their natural properties as antigen-presenting cells, γδ-T cells can engulf TAAs and can be restimulated for further activation and expansion. Subsequently, γδ-T cells secrete TAAs into γδ-T-EVs (TAAs), which contain multiple tumor antigens and can induce a wide-ranging antitumor immune response.

In some embodiments, the vesicles are isolated from primary cells isolated from the subject to be treated. However, the cells can also be isolated from a subject not being treated.

Extracellular vesicles can be isolated using differential centrifugation, flotation density gradient centrifugation, filtration, high performance liquid chromatography, and immunoaffinity-capture. Ultrafiltration and high-performance liquid chromatography (HPLC) are additional methods of isolating EVs based on their size differences. EVs prepared by HPLC are highly purified.

### III. METHODS OF USE

Methods of using the disclosed compositions are also provided. In some embodiments, the methods include contacting cells, or administering to a subject in need thereof, an effective amount of a composition including extracellular vesicles.

The frequency of administration of a method of treatment can be, for example, one, two, three, four or more times daily, weekly, every two weeks, or monthly. In some embodiments, the composition is administered to a subject once every 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days. In some embodiments, the frequency of administration is once, twice or three times weekly, or is once, twice or three times every two weeks, or is once, twice or three times every four weeks. In some embodiments, the composition is administered to a subject 1-3 times, preferably 2 times, a week.

In one aspect, the composition described herein can be administered to a subject comprising a human or an animal including, but not limited to, a mouse, dog, cat, horse, bovine or ovine and the like, that is in need of alleviation or amelioration from a recognized medical condition.

The vesicles and pharmaceutical compositions described herein can be administered to the subject in a number of ways depending on whether local or systemic treatment is desired, and on the area to be treated. Thus, for example, pharmaceutical composition described herein can be administered as an ophthalmic solution and/or ointment to the surface of the eye. Moreover, a pharmaceutical composition can be administered to a subject vaginally, rectally, intranasally, orally, by inhalation, or parenterally, for example, by intradermal, subcutaneous, intramuscular, intraperitoneal, intrarectal, intraarterial, intralymphatic, intravenous, intrathecal and intratracheal routes. Parenteral administration, if used, is generally characterized by injection. Injectables can be prepared in conventional forms, either as liquid solutions or suspensions, solid forms suitable for solution or suspension in liquid prior to injection, or as emulsions. A more recently revised approach for parenteral administration involves use of a slow release or sustained release system such that a constant dosage is maintained. See, e.g., U.S. Pat. No. 3,610,795.

In some embodiments, the compositions are administered systemically, for example, by intravenous or intraperitoneal administration, in an amount effective for delivery of the compositions to targeted cells.

In preferred embodiments, the compositions are administered locally, for example, by injection directly into, or adjacent to, a site to be treated. Typically, local injection causes an increased localized concentration of the compositions which is greater than that which can be achieved by systemic administration.

The dosages or amounts of the active agents described herein are large enough to produce the desired effect in the method by which delivery occurs. The dosage should not be so large as to cause adverse side effects, such as unwanted cross-reactions, anaphylactic reactions, and the like. Generally, the dosage will vary with the age, condition, sex and extent of the disease in the subject and can be determined by one of skill in the art. The dosage can be adjusted by the individual physician based on the clinical condition of the subject involved. The dose, schedule of doses and route of administration can be varied.

Exemplary dosage for in vivo methods are discussed in the experiments below. As further studies are conducted, information will emerge regarding appropriate dosage levels for treatment of various conditions in various patients, and the ordinary skilled worker, considering the therapeutic context, age, and general health of the recipient, will be able to ascertain proper dosing. The selected dosage depends upon the desired therapeutic effect, on the route of administration, and on the duration of the treatment desired.

The disclosed methods of use include co-administration of one or more chemotherapeutic agents. Examples include, but are not limited to Alkylating agents such as Altretamine; Bendamustine; Busulfan; Carboplatin; Chlorambucil; Cisplatin; Cyclophosphamide; Dacarbazine; Ifosfamide; Mechlorethamine; Melphalan; Oxaliplatin; Procarbazine; Temozolomide; Thiotepa; Trabectedin, Carmustine, Lomustine; Streptozocin. Antimetabolites include 5-fluorouracil; 6-mercaptopurine; Azacitidine; Capecitabine; Cladribine; Clofarabine; Cytarabine; Decitabine; Floxuridine; Fludarabine; Gemcitabine; Hydroxyurea; Methotrexate; Nelarabine; Pemetrexed; Pentostatin; Pralatrexate; Thioguanine; Trifluridine/tipiracil combination. Topoisomerase inhibitors include, Etoposide; Irinotecan; Irinotecan liposomal; Mitoxantrone, Teniposide and Topotecan. Mitotic inhibitors include Cabazitaxel, Docetaxel, Nab-paclitaxel, Paclitaxel, Vinblastine, Vincristine, Vinorelbine. Antitumor antibiotics include, Daunorubicin, Doxorubicin, Epirubicin, Idarubicin, Mitoxantrone, Valrubicin, Bleomycin, Dactinomycin, and Mitomycin-C.

The disclosed compositions and methods can be understood by the following non-limiting examples.

### EXAMPLES

### Example 1: Development of γδ-T Cell Extracellular Vesicles-Based Tumor Vaccines with Dual Antitumor Activities

### Methods

### Cell Culture

EBV-LCL cell lines were established using huPBMCs isolated from healthy donors provided by Hong Kong Red Cross as described before (Xiang, et al., Cancer Cell, 26 (4): p. 565-76 (2014)). Briefly, huPBMCs were treated with EBV-containing supernatants of B95.8EbfaV-GFP or B95-8 cell line (Speck and Longnecker, Arch Virol, 144 (6): p. 1123-37 (1999)). (kindly provided by Diane Hayward, Johns Hopkins University, Baltimore) and cultured with 15% fetal bovine serum (FBS)-RPMI-1640 medium supplemented with cyclosporine-A. The EBV-positive gastric carcinoma SNU-719 cell line (Korean cell line bank) was cultured in 10% FBS-RPMI-1640 medium. Practically, γδ-T cells/DCs were cultured for EVs preparation, and EBV-LCL cell lines were induced immediately after huPBMCs isolation. The remaining huPBMCs and autologous transformed EBV-LCL cells were frozen and subjected to establishing tumor-bearing humanized mouse models. All the research protocols and animal work in the present study were approved by The Institutional Review Board of the University of Hong Kong/Hospital Authority Hong Kong West Cluster and the Committee on the Use of Live Animals in Teaching and Research, The University of Hong Kong.

Preparation of γδ-T-EVs and γδ-T-EVs-Based Vaccine γδ-T cells were expanded for EVs isolation through using improved procedures from previous studies (Wang, et al., Sci Transl Med, 12: p. eaaz3426 (2020); Wang, et al., J Immunother Cancer, 10 (2) (2022); Qin, et al., J Virol, 85 (19): p. 10109-16 (2011)). Briefly, huPBMCs were isolated from buffy coats of healthy donors from Hong Kong Red Cross. The huPBMCs were activated with 9 μg/ml pamidronate at day 0 and day 3, and cultured in 10% FBS-RPMI-1640 medium. Human recombinant interleukin-2 (IL-2; Invitrogen) was added from day 3 in a final concentration of 200 IU/ml. After 14-20 days, γδ-T-EVs were prepared using protocols modified from previous studies. γδ-T cells were conditioned in EVs-free culture medium supplemented with 200 IU/ml IL-2, in which FBS-derived EVs had been removed in advance by ultracentrifugation for 18 h at 100,000 g (SW32Ti rotor, Beckman). 48 h later, the conditioned medium was harvested for EVs isolation.

To prepare γδ-T-EVs loaded with tumor-associated antigens, (TAAs), γδ-T cells were pulsed with 500 μg/ml TAAs (which were tumor lysates of EBV-LCL cells obtained via freeze-thaw). 24 h later, γδ-T cells were washed extensively with PBS and cultured in EVs-free culture medium supplemented with 200 IU/ml IL-2.48 h later, the conditioned medium was harvested and subjected to EVs isolation. In the functional assays, equivalent amount of TAAs to that in γδ-T-EVs-based vaccine was used as control after the quantification and normalization via the detection of EBV antigens by western blot.

To prepare γδ-T-EVs (EN1), EBNA1 peptide pools were directly loaded on EVsomes using the methods described before (Mu, et al., Cell Mol Immunol, (8): p. 944-956 (2022)). Briefly, γδ-T-EVs were mixed with peptide pool (Miltenyi) and an equal volume of acetate buffer (pH 5.2) for 30 minutes at 4 °C. After acidification, the preparations were neutralized to pH 7.0 using 2 mol/L Tris buffer (pH 11) on ice for 45 min. The unbound peptides were excluded after filtering through a 100-kDa cut-off filter (Pierce). The same amount of γδ-T-EVs or peptide pool used for peptide loading was filtrated in parallel and the fraction above the filter was collected and served as control.

### Preparation of DC-EVs and DC-EVs-Based Vaccine

To prepare iDCs, human CD14+ cells were isolated from huPBMCs using magnetic beads (Miltenyi Biotec) and incubated for six days in 10%-FBS-RPMI medium supplemented with 10 ng/ml IL-4 and 50 ng/ml GM-CSF (Peprotech) (Wu, et al., BMC Infect Dis, 11: p. 201 (2011)). iDCs cells were pulsed with or without 500 μg/ml TAAs for 10 h. Then, 0.5 μg/ml lipopolysaccharide was added. 14 h later, the cells were washed extensively with PBS and cultured in EVs-free 10% FBS-RPMI culture medium. 48 h later, the conditioned medium was harvested for EVs isolation.

### EVs Isolation and Characterization

EVs were isolated by differential ultracentrifugation at 4 °C as described before (Wang, et al., Sci Transl Med, 12: p. eaaz3426 (2020); Wang, et al., J Immunother Cancer, 10 (2) (2022)). The conditioned medium was centrifuged at 300×g for 10 min, 2,000×g for 10 min and 10,000×g for 30 min. Then, the supernatant was filtered using 0.22-μm syringe filter and ultracentrifuged at 100,000×g for 70 min twice (SW32Ti rotor, Beckman). EVs were dissolved using PBS. The protein concentration of EVs was determined by a BCA Protein Assay Kit (Pierce, Bonn). The size distribution of EVs was determined through nanoparticle tracking analysis. For flow cytometry analysis, EVs were conjugated with 4-μm aldehyde/sulfate latex beads overnight (Thery, et al., Curr Protoc Cell Biol, Chapter 3: p. Unit 3 22 (2006)) and incubated with glycine to block remaining binding sites. The EVs-loaded beads were stained with antibodies and corresponding isotype controls. For Western blot analysis, proteins from cellular lysates or EVs were obtained by lysis in radioimmunoprecipitation assay buffer in the presence of protease inhibitor cocktail (Thermo Fisher Scientific) and separated by SDS-electrophoresis on 8 to 12% gels. Subsequently, proteins were transferred onto membranes and blocked with 5% nonfat milk. Membranes were incubated with anti-Alix (Abcam), anti-EBNA1 (Santa Cruz) or anti-LMP2a antibodies overnight (Abcam). After incubation with corresponding secondary horseradish peroxidase (HRP)-conjugated antibodies, chemiluminescence signals were detected with Immobilon Classico Western HRP substrate (Millipore)

### Induction of Tumor Antigen-Specific T Cells In Vitro

Completed huPBMCs or huPBMCs with HLA-DR+ cells depletion by anti-HLA-DR MicroBeads (Miltenyi) from EBV-seropositive donors were incubated with 20 μg/ml EVs preparations and cultured in RPMI-1640 medium supplemented with 10% human AB serum. After seven days, the cells were restimulated with 1 μg/ml corresponding or irrelevant peptide pools for 6 h, with the addition of 10 μg/ml brefeldin A (BFA, Sigma-Aldrich) 2 h later. The antigen-specific cells were detected on flow cytometry by staining T cell markers and IFN-γ (Biolegend).

### Determination of DCs Maturation and Functions

To determine the effects of EVs-based vaccine on DCs maturation and functions, iDCs were cultured with 20 μg/ml EVs or EVs-based vaccine. In some experiments, neutralizing anti-IFN-γ antibody or isotype antibody was added. 48 h later, the cells were collected to detect maturation markers (e.g., CD40, CD83, CD86 and MHC-II). Cell culture supernatant was also harvested to detect the secretion of IL-6 and TNF-α. A mixed lymphocyte reaction (MLR) was performed to determine the functional activity of DCs as described before (Leung, et al., Blood, 2013.121 (9): p. 1584-94). Briefly, allogeneic CD3 T cells were isolated from huPBMCs using a Pan T cell isolation kit (Miltenyi Biotec) and stained with Carboxyfluorescein succinimidyl ester (CFSE). Then, the EVs preparations-pretreated iDCs were cocultured with allogenic CD3 T cells in a ratio of 1:10. Five days later, T cell proliferation was measured by flow cytometry. The culture supernatant of MLR assay was also collected and subjected to detecting the secretion IFN-γ.

### Cell Apoptosis and Proliferation Assay

EBV-LCL or SNU-719 cells were treated with 30 μg/ml EVs or EVs-based vaccine. 18-24 h later, the cell apoptosis was evaluated through annexin V expression, and the cell proliferation was assessed through Ki67 expression (Biolegend).

### Cytotoxicity Assay

As effector cells, T cells were negatively selected from γδ-T-EVs or γδ-T-EVs-based vaccine pretreated huPBMCs using Pan T cell isolation kit (Miltenyi). The cytotoxic activities of T cells were determined against the following targets: (i) autologous EBV-LCL, (ii) autologous EBV-LCL pulsed with 1 μg/ml EBNA1, LMP2a or HIV p17 peptides pools. Effector and target cells were cocultured at a ratio of 10:1. To distinguish both cell populations in detection, target cells were stained with CFSE before coculturing. After 6 h, the cells were stained with propidium iodide (PI) and then analyzed by flow cytometry. The death of target cells was measured as the percentage of PI+ cells in the CFSE-positive population.

### Establishment and Treatment of EBV-Associated Tumors in Rag2-/-γc-/- and Humanized Mice

Rag2-/-γc-/-mice were maintained in the Laboratory Animal Unit of the University of Hong Kong. Four to six-week-old Rag2-/-γc-/-mice were used to build humanized mice by reconstitution with EBV-seropositive CD3 T cells-depleted huPBMCs or complete huPBMCs using a method described previously. After huPBMCs injection, symptoms of graft-versus-host disease (e.g. weight loss, diarrhea) were monitored daily. Generally, the weight loss occurred from day 1 to day 10 and became normal 15-20 days after the huPBMCs injection. Four weeks after huPBMCs reconstitution, the mice were referred to “humanized” mice as they had a functional human peripheral immune system (Tu, et al., J Exp Med, 2011.208 (7): p. 1511-22; Xiang, et al., Cancer Cell, 2014.26 (4): p. 565-76). Rag2-/-γc-/-mice or humanized mice were implanted s.c. with SNU-719 or EBV-LCL cells to establish EBV-associated tumor models. For humanized mouse model, EBV-LCL cells were autologous to the huPBMCs. Almost all tumor cells-injected mice developed tumors within two weeks if no treatment was given. Mice with palpable tumors were randomly grouped for therapeutic experiments. Mice were age- and gender-matched, and there were no evident differences in tumor volume among the groups before treatment. The mice were i.p. injected with autologous, allogenic EVs or EVs-based vaccine as indicated. Tumor volume, tumor incidence and animal survival were monitored and calculated at indicated time. Tumor volume was determined by a digital caliper and calculated as length×[width]2×0.52. Comply with the regulation of the University of Hong Kong, mice bearing subcutaneous tumors with diameters reaching 17 mm were euthanized. Tumor tissues were harvested for immunohistochemical analysis.

### Preventive Effects of γδ-T-EVs-Based Vaccine Against EBV-Associated Tumors in Humanized Mice

Humanized mice were established as indicated above. The mice were vaccinated with γδ-T-EVs or γδ-T-EVs (TAAs). Two weeks later, animals were boosted with the same preparations. Seven days after booster vaccination, EBV-LCL cells (1×10⁶ per mouse) were inoculated into the mice. Tumor incidence, tumor volume and mice survival were calculated at the indicated time. Tumor development was also validated by an IVIS Spectrum in vivo imaging system (Caliper Life Sciences).

### Detection of Tumor-Specific T Cells Ex Vivo

50 μg/mouse γδ-T-EVs or γδ-T-EVs (TAAs) were injected (i.p.) into EBV-induced B cell lymphoma-bearing humanized mice biweekly for two doses. Equivalent amount of TAAs was also used as control. Seven days post the booster injection, the peripheral blood nucleated cells were isolated and subjected to peptide restimulation to detect EBNA1- or LMP2a-specific T cells.

### Histological and Immunohistochemical Analysis

Tumor tissues were fixed with 10% formalin and embedded in paraffin for sectioning. The sections were subjected to immunohistochemistry staining. Ki67 was detected by immunohistochemistry using an anti-human Ki67 antibody (Abcam, UK) and visualized by a diaminobenzidine detection kit (Maixin, China).

### Flow Cytometric Analysis

Surface staining of cells was performed using the following antibodies: anti-CD63 (H5C6), anti-CD3 (HIT3a), anti-CD4 (RPA-T4), anti-CD8 (SK1), anti-MHC-II (Tü39), anti-CD83 (HB15e), anti-CD86 (GL-1) and anti-CD40 (5C3). For the intracellular staining, cells were fixed, permeabilized and followed by staining with anti-IFN-γ (B27) or isotype control as described before (Ni, et al., Am J Respir Cell Mol Biol, 58 (6): p. 684-695 (2018)). All samples were detected using a FACSLSR II Flow Cytometer (BD, USA) and analyzed with FlowJo software (Tree Star, USA).

### Cytokine Secretion Assay

The concentrations of cytokines in the culture supernatants were detected and analyzed with LEGENDplex human Cytokine detection kit (BioLegend).

### Statistics

Quantitative data were expressed as mean±SEM. The comparison of two groups was Mann-conducted by the Whitney U test. One-way analysis of variance (ANOVA) with Bonferroni correction was used to compare multiple groups. Two-way ANOVA analysis was used to determine the effects of multiple variables and corresponding interactions. Animal survival and tumor incidence were analyzed via Kaplan-Meier log-rank test. Statistical significance was calculated using two-tailed tests, and P<0.05 was considered significant. Specific sample size, P value, and other details for each test are described in the main text or figure legends.

### Results

### γδ-T-EVs-Based Vaccine Efficiently Induces Tumor-Specific T Cells

EVs were isolated from different donor-derived γδ-T cells through differential ultracentrifugation. Generally, 4.1±0.3 μg γδ-T-EVs proteins could be obtained from 10⁶ cells (N=4). To prepare the vaccine regimens, γδ-T-EVs were directly loaded with tumor antigen EBNA1 (γδ-T-EVs (EN1)) or indirectly loaded with TAAs (γδ-T-EVs (TAAs)) after pulsing γδ-T cells before EV isolation (Damo, et al., Sci Rep, 2015.5: p. 17622; Bu, et al., J Mol Neurosci, 2015.56 (3): 631-43).

Characterization analysis revealed that the antigen-loading procedures did not change the size distribution and expression of EVs markers on the γδ-T-EVs-based vaccines (FIGS. 1A-1B). In addition, the γδ-T-EVs (TAAs) contained intact tumor antigens, including EBV antigens such as Epstein-Barr nuclear antigen 1 (EBNA1) and latent membrane protein 2a (LMP2a) (FIG. 1C), which are critical therapeutic targets of EBV-associated tumors (Cui, and Snapper, Front Immunol, 2021.12: p. 734471). These data indicate that TAAs can be loaded on γδ-T-EVs using an indirect-antigen loading strategy.

The results showed that both γδ-T-EVs (EN1) induced more antigen-specific CD4 and CD8 T cells than other groups. The control of free peptide removal did not enhance the antigen-specific T-cell responses, suggesting the efficient removal of the free peptide by the filtration procedure after peptide loading on γδ-T-EVs (FIG. 1D-1E). In addition, preloading of tumor peptides on γδ-T-EVs significantly increased the efficacy to induce tumor-specific T cells responses than that treated free peptide plus γδ-T-EVs without preloading. In addition, γδ-T-EVs (TAAs) induced more EBNA1- and LMP2a-specific CD4 and CD8 T cells compared with γδ-T-EVs, TAAs or γδ-T-EVs+free TAAs (FIG. 1F-1G). The cells treated with the γδ-T-EVs-based vaccines did not respond to irrelevant HIV p17 peptides, confirming the antigen specificity of these T-cell responses (FIG. 1D-1G). Taken together, these data indicate that γδ-T-EVs can be designed as tumor.

### γδ-T-EV Vaccines Amplify Tumor-Specific T-Cell Immunity

### Yò-T-EVs Displays Immune Adjuvant Effects on Antigen-Presenting Cells

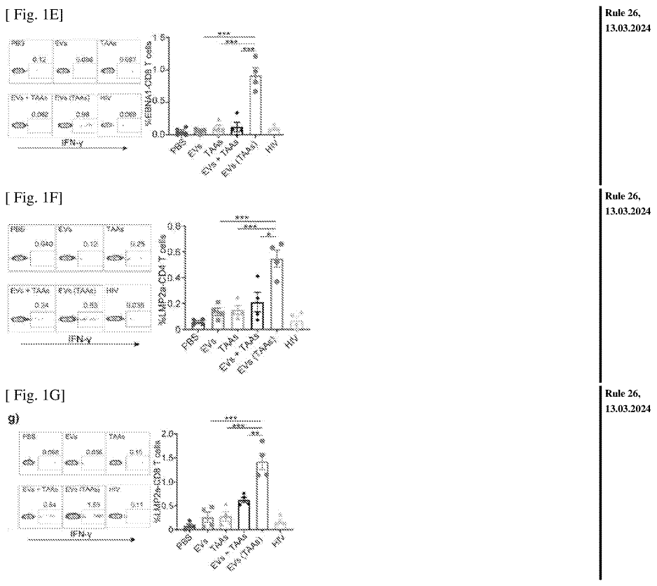

To determine whether recipient APCs could influence the efficacy of γδ-T-EVs-based vaccines, anti-HLA-DR microbeads were used to deplete the APCs in huPBMCs as previously described (Walker, et al., J Immunol, 2009.182 (3): p. 1548-59). As shown in FIG. 2A-2B, HLA-DR+ cells-depleted huPBMCs had much fewer antigen-specific T cells induced by the γδ-T-EVs-based vaccines than complete huPBMCs, suggesting that the efficacy of γδ-T-EVs-based vaccines depended on recipient APCs. Thus, hypothetically, the γδ-T-EVs-based vaccines could mobilize APCs to induce antigen-specific T-cell responses.

To confirm this hypothesis, purified CD3+ T cells were treated with the preparations in the absence or presence of immature DCs (iDCs). γδ-T-EVs (TAAs) cocultured with iDCs increased the efficacy of γδ-T-EVs (TAAs) to induce tumor-specific T cell responses (FIG. 2C). Although γδ-T-EVs+free TAAs also showed a trend in the augment of tumor-specific T cells in the presence of iDCs, its efficacy was significantly lower than γδ-T-EVs preloaded with TAAs (γδ-T-EVs (TAAs)) (FIG. 2C). Hence, co-delivery of antigen and γδ-T-EVs to recipient iDCs is important to promote tumor-specific T cell responses. Taken together, these findings demonstrate that γδ-T-EVs-based vaccine can induce tumor-specific T cells through recipient APCs.

To elucidate the effects of γδ-T-EVs on APCs, iDCs were cultured with TAAs, γδ-T-EVs, or γδ-T-EVs (TAAs) for 48 h. Compared with TAAs, both of γδ-T-EVs and γδ-T-EVs (TAAs) promoted the expression of DCs maturation makers, including MHC-II, CD83, CD86 and CD40 (FIG. 2D-2G). In addition, the secretion of pro-inflammatory cytokine IL-6 and TNF-α from iDCs was significantly increased after being treated with γδ-T-EVs or γδ-T-EVs (TAAs) (FIG. 2H-2I). Moreover, iDCs pretreated with γδ-T-EVs or γδ-T-EVs (TAAs) had higher antigen-presenting abilities, as revealed by promoting more proliferation (FIG. 2J) and IFN-γ secretion (FIG. 2K) of allogenic CD3+ T cell in a mixed lymphocyte reaction (MLR) assay. These data suggested that γδ-T-EVs could promote iDCs maturation and enhance their antigen-presenting abilities, which was also evidenced by the induction of dendrites from iDCs by γδ-T-EVs or γδ-T-EVs-based vaccine. In addition, the effects of γδ-T-EVs (TAAs) on iDCs were comparable to γδ-T-EVs (FIG. 2L), suggesting that the antigen loading procedures did not hamper the adjuvant effects of γδ-T-EVs (TAAs).

The underlying mechanism of γδ-T-EVs-induced adjuvant effects on DCs was further determined. Similar to their parental cells, γδ-T-EVs expressed robust IFN-γ, which is a well-known cytokine to promote DCs maturation. The blockage of IFN-γusing neutralizing antibody inhibited the antigen-presenting abilities of iDCs enhanced by γδ-T-EVs (FIG. 2L), indicating that the carried IFN-γ mediated the immune adjuvant effects of γδ-T-EVs. Taken together, these results demonstrate that γδ-T-EVs-based vaccine can promote tumor-specific T cell responses by delivering antigens and serving as an immune adjuvant on recipient APCs.

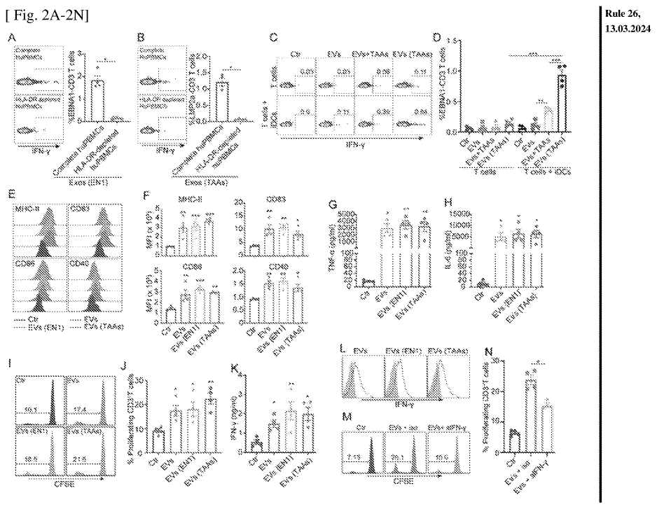

γδ-T-EVs-based vaccine preserves direct antitumor activities in vitro and in vivo Subsequent studies determined whether the γδ-T-EVs-based vaccines inherited this direct antitumor property. As shown in FIGS. 3A and 3B, both γδ-T-EVs and γδ-T-EVs (TAAs) induced more tumor cell apoptosis compared with TAAs. No significant differences were found between γδ-T-EVs or γδ-T-EVs (TAAs) (FIG. 3A-3B).

To determine the direct antitumor effects of the γδ-T-EVs-based vaccine in vivo, EBV-induced B cell lymphoma-bearing Rag2-/-γc-/-mice were treated with γδ-T-EVs, or γδ-T-EVs (TAAs) (FIG. 3C). Mice treated with γδ-T-EVs or γδ-T-EVs (TAAs) effectively inhibited tumor growth and prolonged mice survival when compared with TAAs (FIG. 3D-3E). No significant differences in their antitumor effects were observed between γδ-T-EVs or γδ-T-EVs (TAAs) (FIG. 3D-3E). The tumor cells in mice treated with γδ-T-EVs or γδ-T-EVs (TAAs) were found with lower proliferative capability because immunophenotypic analysis found fewer Ki-67 positive cells in residual tumors.

Other tumor models based on EBV-associated gastric carcinoma or nasopharyngeal carcinoma were also used to validate the direct antitumor activities of the γδ-T-EVs-based vaccine in vivo (FIG. 3F). Similarly, smaller tumor volume and longer survival of tumor-bearing mice were found after being treated with γδ-T-EVs or γδ-T-EVs (TAAs) than TAAs (FIG. 3G-3H). Consistently, no significant differences were found between γδ-T-EVs or γδ-T-EVs (TAAs) (FIG. 3G-3H). The tumor cells in mice treated with γδ-T-EVs or γδ-T-EVs (TAAs) were also found with lower proliferative capability, as fewer Ki-67 positive cells were found in residual tumors by histological analysis. Consistent antitumor effects of γδ-T-EVs-based vaccines were also identified on NPC tumors (FIG. 3I-3K). Taken together, the data demonstrate that γδ-T-EVs-based vaccine preserves their direct antitumor activities both in vitro and in vivo.

### γδ-T-EVs-Based Vaccine Inhibits Tumor Progression in Humanized Mice

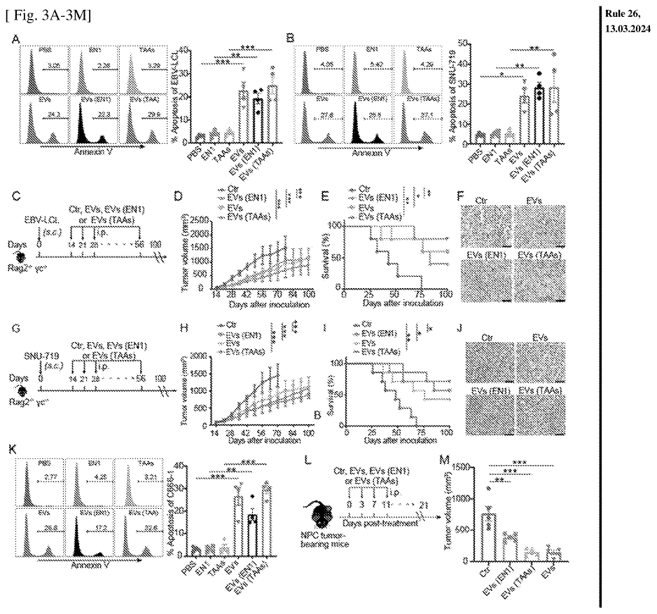

Humanized mice harboring functional human immune cells (e.g., B cells, T cells and antigen-presenting cells) are especially appropriate for preclinical modeling of vaccine therapy. To determine the therapeutic efficacy of γδ-T-EVs-based vaccine in humanized mice, tumor models was built as previously described, through s.c. inoculation of autologous EBV-LCL in the humanized mice. Mice that had developed subcutaneous tumors were treated with γδ-T-EVs or γδ-T-EVs-based vaccines as indicated (FIG. 4A). γδ-T-EVs or γδ-T-EVs-based vaccines inhibited tumor growth and prolonged mice survival compared with control group (FIG. 4B-4C). The tumor cells in mice treated with γδ-T-EVs or γδ-T-EVs-based vaccines were also found with lower proliferative capability, as fewer Ki-67 positive cells were identified in corresponding residual tumors by histological analysis. In addition, different from their comparable therapeutic efficacies in immunodeficient mice (FIG. 3C-3H), γδ-T-EVs-based vaccines had higher therapeutic efficacy than γδ-T-EVs in humanized mice (FIG. 4B-4C). Interestingly, γδ-T-EVs (TAA) had higher therapeutic efficacy than γδ-T-EVs (EN1) (FIG. 4B-4C), suggesting that the tumor-specific T cell responses induced by γδ-T-EVs (TAAs) might be more potent than those induced by γδ-T-EVs (EN1) to elucidate tumor cells.

Consistent with in vitro findings, both γδ-T-EVs (EN1) and γδ-T-EVs (TAAs) induced tumor-specific T-cell responses in humanized mice. However, γδ-T-EVs (TAAs) induced antigen-specific T cell responses against multiple tumor proteins (e.g. EBNA1, LMP2a), while γδ-T-EVs (EN1) only targeted EBNA1 antigen (FIG. 4D-4E). Besides, data showed that the tumor-specific T cells induced by γδ-T-EVs (TAA) had higher cytotoxic effects than those induced by γδ-T-EVs (EN1) against autologous EBV-LCL. In addition, preloading of EBNA1 or LMP2a, rather than HIV p17 peptides on autologous EBV-LCL resulted in higher cytotoxic activities (FIG. 4F-4G). These data show that γδ-T-EVs-based vaccine carrying tumor lysates can induce antigen-specific T cells against multiple tumor targets and display higher therapeutic efficacy against EBV-associated tumors. Therefore, γδ-T-EVs (TAAs) were further investigated in the following study.

### Allogeneic γδ-T-EVs-Based Vaccine Mobilizes Recipient APCs to Induce Tumor-Specific T-Cell Responses

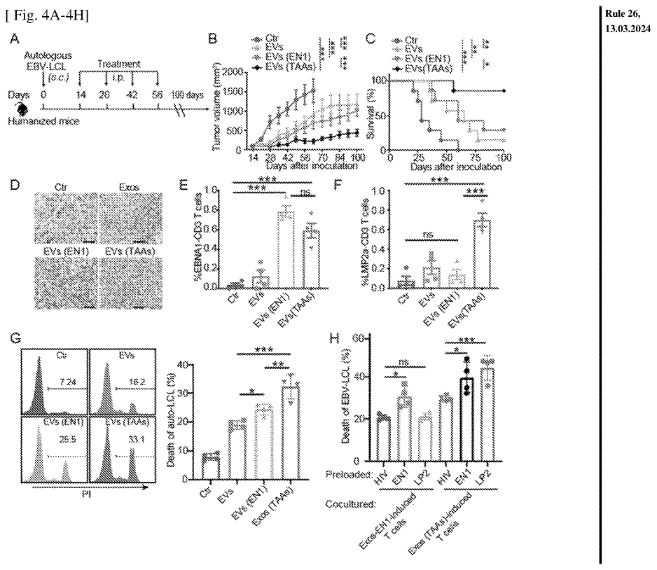

Since many cancer patients are immunocompromised either because of cancer or anti-cancer therapy, it is very difficult to obtain enough patient γδ-T cells and expand them ex vivo to prepare autologous γδ-T-EVs in clinical practice. In addition, collecting γδ-T cells and preparing γδ-T-EVs from allogeneic healthy donors are much easier and have advantages in standardization and centralization. Therefore, additional studies further determined the antitumor potentials of allogeneic γδ-T-EVs-based vaccine. As shown in FIG. 5A-5B, autologous and allogeneic γδ-T-EVs or γδ-T-EVs (TAAs) promoted iDCs maturation, as revealed by upregulation of CD83 and MHC-II compared with TAAs treatment. The secretion of pro-inflammatory cytokines (FIG. 5C-5D) and antigen-presenting abilities of iDCs were also comparable after being treated with autologous versus allogeneic γδ-T-EVs or γδ-T-EVs-based vaccine. These data demonstrate that allogeneic

γδ-T-EVs-Based Vaccine has Similar Immune Adjuvant Effects with Autologous Counterparts

The efficacy of autologous versus allogeneic γδ-T-EVs-based vaccine in the induction of tumor-specific T cells was further compared. Compared with γδ-T-EVs, both autologous and allogeneic γδ-T-EVs (TAAs) induced more tumor-specific T cells, in which the levels of tumor-specific T cells were comparable (FIG. 5F-5G). Similarly, depletion of HLA-DR+APCs from huPBMCs prevented the induction of tumor-specific T cells by both autologous and allogeneic γδ-T-EVs (TAAs) (FIG. 5H), suggesting that recipient APCs are indispensable for the functions of both autologous and allogeneic γδ-T-EVs-based vaccines. In summary, these results indicate that allogeneic γδ-T-EVs-based vaccine is comparable with its autologous counterpart in mobilizing recipient APCs and inducing tumor-specific T-cell responses.

### Allogeneic γδ-T-EVs-Based Vaccine Inhibits Tumor Progression in Humanized Mice

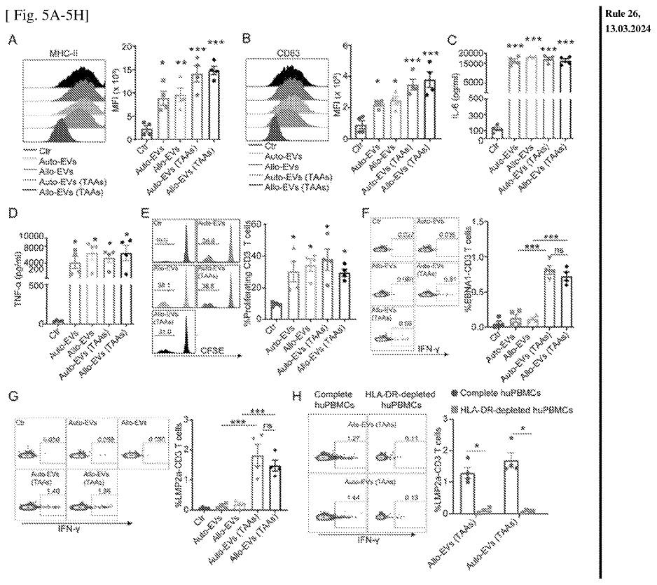

The antitumor efficacies of allogeneic versus autologous γδ-T-EVs-based vaccine in humanized mice were further compared. As indicated in FIG. 6A, EBV-induced B cell lymphoma-bearing humanized mice were treated with allogeneic or autologous γδ-T-EVs (TAAs). PBS, TAAs or γδ-T-EVs was used as controls. Mice treated with γδ-T-EVs had smaller tumor growth and longer mice survival than either PBS or TAAs treatment. After being loaded with tumor antigens, either autologous or allogeneic γδ-T-EVs (TAAs) showed better antitumor efficacies than γδ-T-EVs (FIG. 6B-6C). In addition, allogeneic γδ-T-EVs (TAAs) was not inferior to autologous γδ-T-EVs (TAAs) in tumor control (FIG. 6B-6C). The tumor cells in mice treated with either autologous or allogeneic γδ-T-EVs (TAAs) were also found with lower proliferative capability, as fewer Ki-67 positive cells were found in corresponding residual tumors. Considering the advantages of allogeneic EVs in clinical application, these data suggest the great translational potentials of allogeneic γδ-T-EVs-based vaccine in cancer therapy.

To evaluate whether human T cells are involved in the antitumor activities of allogeneic γδ-T-EVs-based vaccine, humanized mice reconstituted with complete huPBMCs, or CD3 T cell-depleted huPBMCs were used for tumor establishment and treatment (FIG. 6D). Consistently, treatment of complete huPBMCs-reconstituted humanized mice with allogeneic γδ-T-EVs (TAAs) significantly suppressed tumor growth and prolonged mice survival when compared with TAAs (FIG. 6E-6F). However, the therapeutic efficacy was decreased in the humanized mice reconstituted with CD3 T cell-depleted huPBMCs. Moreover, after being treated with allogeneic γδ-T-EVs (TAAs), the tumor cells in mice reconstituted with CD3 T cell-depleted huPBMCs had increased proliferative capability than those in the mice reconstituted with complete huPBMCs, as more Ki-67 positive cells were found in corresponding residual tumors. Therefore, these results indicate that allogeneic γδ-T-EVs-based vaccine controls tumor progression in humanized mice.

### Allogeneic γδ-T-EVs-Based Vaccine Prevents Tumor Development in Humanized Mice

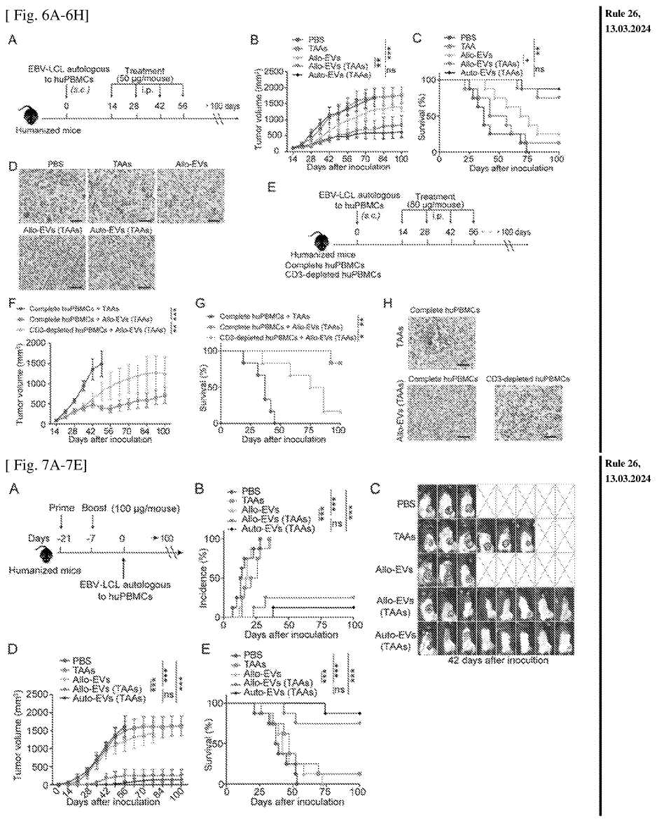

To determine whether the allogeneic γδ-T-EVs-based vaccine can prevent tumor development, humanized mice were injected with PBS, TAAs, allogeneic γδ-T-EVs or γδ-T-EVs (TAAs) i.p. at day 21 and 7 before tumor cell inoculation. Autologous γδ-T-EVs (TAAs) also served as a control (FIG. 7A). One week after the booster injection, the mice were challenged with autologous EBV-LCL cells (FIG. 7A). Rapid development of tumor was observed in all the mice immunized with PBS, TAAs or γδ-T-EVs. However, only 25% and 12.5% of mice had developed tumors after being immunized with allogeneic and autologous γδ-T-EVs (TAAs), respectively (FIG. 7B). In Vivo Imaging analysis was also used to validate the tumor development of each group at six weeks after tumor cell inoculation. In addition, immunization with allogeneic or autologous γδ-T-EVs (TAAs) effectively inhibited tumor growth (FIG. 7C) and prolonged mice survival (FIG. 7D). The preventive efficacies of allogeneic and autologous γδ-T-EVs (TAAs) had no significant differences (FIG. 7B-7D). Thus, our data demonstrate that allogeneic γδ-T-EVs-based vaccine has preventive effects against tumor development.

Allogeneic γδ-T-EVs-based vaccine displays advantages over DC-EVs-based vaccine through dual antitumor activities

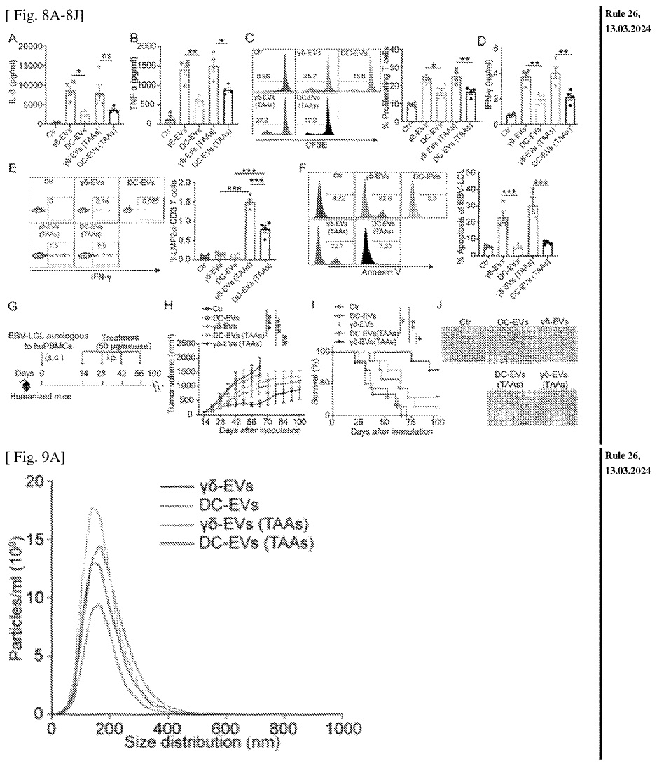

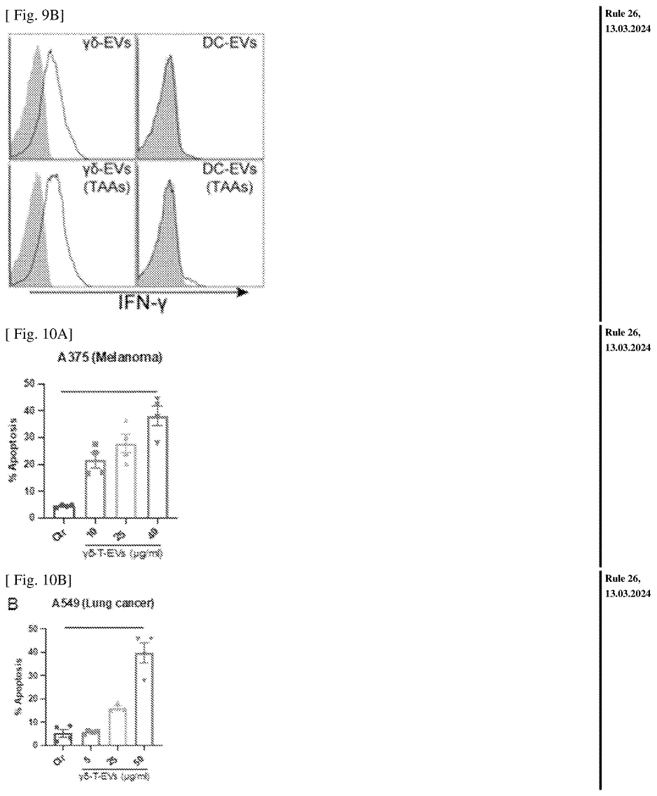

As allogeneic DC-EVs also have adjuvant effects and can be used as a nanocarrier in tumor vaccine, the efficacies of allogeneic γδ-T-EVs-with DC-EVs-based vaccines were then compared. Both vaccines were prepared by pulsing parental cells with TAAs, before EVs isolation. FIG. 9A is a graph of size distribution of DC-EVs- and γδ-T-EVs-based vaccines. Size distribution of γδ-T-EVs, DC-EVs, γδ-T-EVs (TAAs) or DC-EVs (TAAs) determined by ZetaView Particle Tracking Analyzer. Data was shown from one of three independent experiments. T-T-EVs- and DC-EVs-based vaccines had similar size distribution profiles with peaks ~160 nm (FIG. 9B). In addition, γδ-T-EVs and γδ-T-EVs (TAAs) carried robust IFN-γ, while DC-EVs and DC-EVs (TAAs) did not. Both allogeneic DC-EVs- and γδ-T-EVs-based vaccines induced dendrites from iDCs. However, allogeneic γδ-T-EVs and γδ-T-EVs (TAAs) induced more pro-inflammatory cytokines from iDCs than either DC-EVs or DC-EVs (TAAs) (FIG. 8A-8B). In a MLR assay, allogeneic γδ-T-EVs- and γδ-T-EVs (TAAs)-pretreated iDCs also promoted more T cell proliferation and IFN-γ secretion than the DC-EVs counterparts (FIG. 8C-8D). These data suggest that the allogeneic γδ-T-EVs-based vaccine has higher immune adjuvant effects than DC-EVs-based vaccine.

The vaccine efficacies of allogeneic DC-EVs- and γδ-T-EVs-based vaccines were further compared. TAAs, allogeneic γδ-T-EVs, γδ-T-EVs (TAAs), DC-EVs or DC-EVs (TAAs) were used to treat huPBMCs. Compared with TAAs or the allogeneic EVs, both allogeneic γδ-T-EVs (TAAs) and DC-EVs (TAAs) induced more tumor-specific T cells (FIG. 8E). However, allogeneic γδ-T-EVs (TAAs) were more effective to induce tumor-specific T cells than allogeneic DC-EVs (TAAs) (FIG. 8E). More importantly, γδ-T-EVs and γδ-T-EVs (TAAs) could directly induce tumor cell apoptosis, while DC-EVs or DC-EVs (TAAs) did not (FIG. 8F). These data suggest that γδ-T-EVs-based vaccine has advantages over DC-EVs-based vaccine by displaying dual antitumor activities.

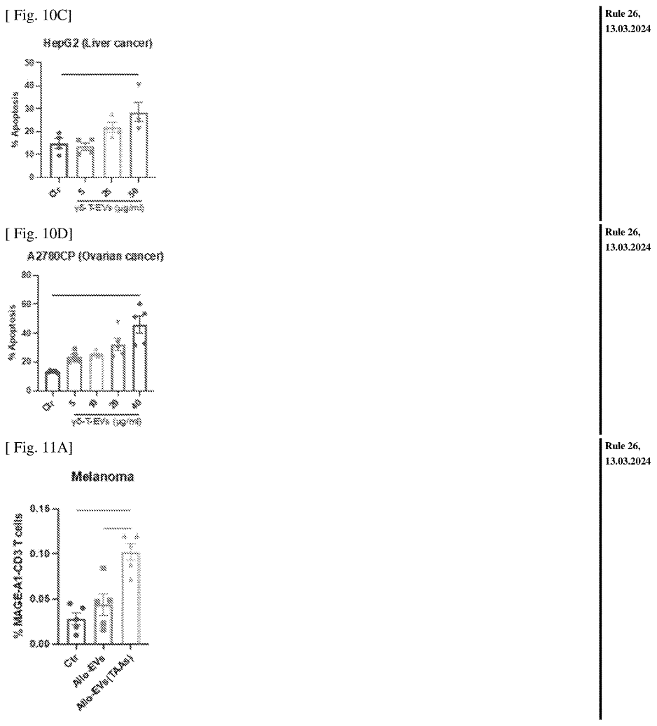

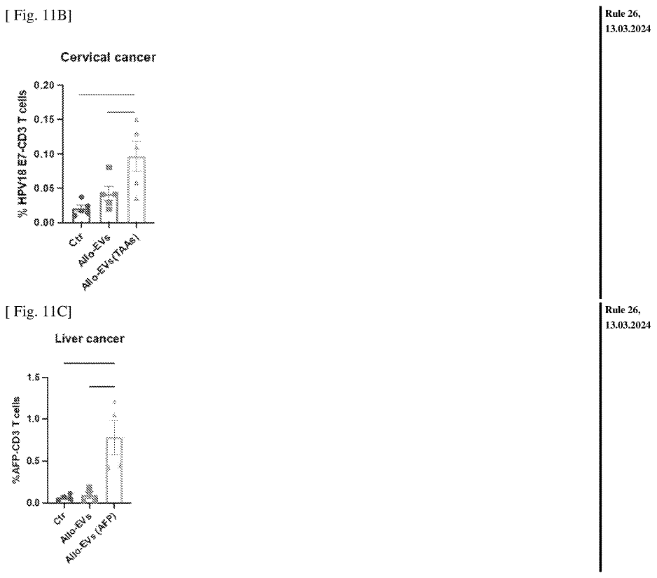

The therapeutic efficacies of allogeneic γδ-T-EVs-versus DC-EVs-based vaccines in humanized mice were further compared. As indicated in FIG. 8G, EBV-induced B cell lymphoma-bearing humanized mice were treated with allogeneic γδ-T-EVs (TAAs) or DC-EVs (TAAs), respectively. TAAs or the allogeneic EVs were used as controls. Mice treated with allogeneic γδ-T-EVs (TAAs) or DC-EVs (TAAs) had smaller tumor sizes and longer mice survival than TAAs. However, allogeneic γδ-T-EVs (TAAs) exerted higher therapeutic effects than allogeneic DC-EVs (TAAs) (FIGS. 8H-8I). The proliferation of tumor cells in residual tumors also supported these findings. Thus, our results demonstrate that allogeneic γδ-T-EVs-based vaccine displays advantages over allogeneic DC-EVs-based vaccine in inhibiting tumor progression in humanized mice. Besides EBV-associated tumors, FIGS. 10A-10D and 11A-11C show more examples of γδ-T-EVs loaded with other tumor antigens, such as lung cancer, liver cancer, melanoma, cervical cancer and ovarian, and showed that γδ-T-EVs-based vaccines can kill multiple tumor cells and further induce tumor antigen-specific T cells against multiple tumors.

### SUMMARY

The present study showed that γδ-T-EVs could serve as nanocarriers to deliver TAAs and function as an immune adjuvant to induce antigen-specific T-cell responses. In addition, γδ-T-EVs-based vaccine preserved direct killing activity against tumors. Administration of γδ-T-EVs-based vaccine effectively controlled different tumors in vivo. More importantly, allogeneic γδ-T-EVs-based vaccine also had robust therapeutic effects and displayed advantages over allogeneic DC-EVs-based vaccine by exerting dual antitumor activities. Therefore, the application of γδ-T-EVs-based vaccine has excellent potential in cancer therapy.

As a bridge between innate and adaptive immune systems, γδ-T cells play critical roles in the regulation of APCs function (Ismaili, et al., Clin Immunol, 2002.103 (3 Pt 1): p. 296-302). Here, the studies demonstrated for the first time that γδ-T cells could promote APCs maturation in a cell-free manner through EVs, which was mediated by the IFN-γcarried by γδ-T-EVs. The adjuvant effects of γδ-T-EVs are potent enough without the need of additional adjuvants for γδ-T-EVs-based vaccine, in which the manufacturing processes are generally different from TEVs-based or DC-EVs-based vaccine (Naseri, et al., Oncoimmunology, 2020.9 (1): p. 1779991; Xia, et al., Biomed Pharmacother, 2022.152: p. 113250). Therefore, preparing the γδ-T-EVs-based vaccine could be more convenient since preloading the immune adjuvant is unnecessary. Moreover, compared with TEVs-based vaccine, γδ-T-EVs-based vaccine may have a better safety profile due to their nature in tumor surveillance, while TEVs can drive tumorigenesis (Whiteside, Adv Clin Chem, 2016. 74: p. 103-41).

Preparing personalized vaccine for cancer patients is very difficult in clinical practice since the procedures are time- and labor-consuming. In addition, the γδ-T cells of cancer patients are difficult to be expanded ex vivo for EVs production due to the immune dysregulation caused by cancer or anti-cancer therapy (e.g., radiotherapy and chemotherapy). In contrast, it is much easier to prepare γδ-T cells from healthy donors in large-scale using the protocols modified by our or others' groups (Alexander, et al., Clin Cancer Res, 2008. 14 (13): p. 4232-40; Bonneville and Scotet, Curr Opin Immunol, 2006. 18 (5): p. 539-46; Kouakanou, et al., Cell Mol Immunol, 2020. 17 (5): p. 462-473; Qin, et al., J Infect Dis, 2012. 205 (11): p. 1646-53; Qin, et al., J Infect Dis, 2009. 200 (6): p. 858-65; Xiang, et al., Cancer Cell, 2014. 26 (4): p. 565-576; Peters, et al., Cell Mol Immunol, 2022. 19 (10): p. 1198-1200), which can ensure the production of γδ-T-EVs for clinical application. The date showed that allogeneic γδ-T-EVs also had strong immune adjuvant effects by inducing bystander activation of recipient APCs. When serving as a nanocarrier of tumor antigens, allogeneic γδ-T-EVs are comparable to their autologous counterparts in the induction of tumor-specific immune responses and tumor control. More importantly, allogeneic γδ-T-EVs-based vaccine not only showed potent therapeutic antitumor effects, but also effectively prevented tumor development. After two doses of immunization with γδ-T-EVs-based vaccine, most mice were protected from tumor development and survived longer. Therefore, γδ-T-EVs-based vaccine has a wide range of application scenarios in clinical practice for preventing and treating tumors. Some previous studies showed that allogenic and syngeneic DC-based vaccines had similar antitumor efficacies, supporting our findings. Considering that preparing γδ-T-EVs from healthy donors has advantages in quality control, standardization and centralization, the allogeneic γδ-T-EVs-based vaccine could be used as an “off-the-shelf” approach in cancer therapy.

DC-EVs-based vaccines have been widely investigated for cancer immunotherapy and demonstrated with promising therapeutic potentials in preclinical models. However, their therapeutic efficacies were limited in clinical trials (Besse, et al., Oncoimmunology, 2016. 5 (4): p. e1071008; Escudier, et al., J Transl Med, 2005. 3 (1): p. 10). The heterogeneity of ex vivo expanded DCs may partially account for the poor therapeutic outcomes of DC-EVs-based vaccines since the EVs derived from immature DCs exerted immunoinhibitory activities. On the contrary, homogeneous and immunostimulatory human γδ-T cells can be expanded in large-scale by phosphoantigens, which ensure the preparation of γδ-T-EVs-based vaccine for clinical use. Similar to our findings, allogeneic DC-EVs were demonstrated with obvious adjuvant effects. Natural γδ-T-EVs had higher adjuvant effects than DC-EVs and induced tumor-specific T-cell responses more effectively after antigen loading. Besides, the γδ-T-EVs-based vaccine preserved direct antitumor activities, while the DC-EVs-based vaccine could not directly kill tumor cells. Therefore, γδ-T-EVs-based vaccine has advantages over DC-EVs-based vaccine via their dual antitumor activities.

In summary, the present provides a strong preclinical proof-of-concept to use γδ-T-EVs-based vaccine in cancer therapy. As a cell-free method, γδ-T-EVs-based vaccine has dual antitumor activities. More importantly, allogeneic γδ-T-EVs-based vaccine also has robust antitumor effects. Therefore, this approach has promising translational potential.

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise.

Throughout the description and claims of this specification, the word “comprise” and variations of the word, such as “comprising” and “comprises,” means “including but not limited to,” and is not intended to exclude, for example, other additives, components, integers or steps.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. It should be understood that all of the individual values and sub-ranges of values contained within an explicitly disclosed range are also specifically contemplated and should be considered disclosed unless the context specifically indicates otherwise. Finally, it should be understood that all ranges refer both to the recited range as a range and as a collection of individual numbers from and including the first endpoint to and including the second endpoint. In the latter case, it should be understood that any of the individual numbers can be selected as one form of the quantity, value, or feature to which the range refers. In this way, a range describes a set of numbers or values from and including the first endpoint to and including the second endpoint from which a single member of the set (i.e. a single number) can be selected as the quantity, value, or feature to which the range refers. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Those skilled in the art will recognize, or be able to ascertain using no more than routine experimentation, many equivalents to the specific embodiments of the method and compositions described herein. Such equivalents are intended to be encompassed by the following claims.

1. An immunogenic composition comprising extracellular vesicles (EVs) derived from γδ-T cells (γδ-T-EVs) in a pharmaceutically acceptable a carrier.

2. The composition of claim 1, further comprising with one or more tumor antigens (TAs).

3. The composition of claim 1, wherein the one or more tumor antigens are selected from the group consisting of Epstein-Barr nuclear antigen 1 (EBNA1) and latent membrane protein 2a (LMP2a).

4. The composition of claim 1, wherein the tumor antigen is selected from the group consisting of Immature laminin receptor, BING-4, Calcium-activated chloride channel 2, Cyclin-B1, Carcinoembryonic antigen; breast cancer antigen; cyclin-dependent kinase-4; chronic myelogenous leukemia (antigen) 66; cancer testis; human papilloma virus E6/E7; epithelial cell adhesion molecule; immunoglobulin; melanoma antigen recognized by T cells-1/-2; melanocortin-1-receptor; stomach cancer-associated protein tyrosine phosphatase-1; tumor antigen-72; T cell receptor; transforming growth factor-β receptor II; tyrosinase-related protein, 9D7, Ep-CAM, EphA3, Her2/neu, Telomerase, Mesothelin, Survivin, NY-ESO-1/LAGE-1, PRAME, SSX-2, Melan-A/MART-1, Gp100/pmel17, Tyrosinase, P. polypeptide, Prostate-specific antigen, β-catenin, BRCA1/2, Fibronectin; p53; Ras and Mucin short variant S1.

5. The composition of claim 1, wherein the tumor antigen is selected from the group consisting of Alpha-fetoprotein (AFP); MAGE (Melanoma-Associated Antigen); Glypican-3 (GPC3); SOX2; WT1 (Wilms Tumor 1); HMGA2 (High Mobility Group AT-Hook 2); HSP90 (Heat Shock Protein 90); PSMA (Prostate-Specific Membrane Antigen); CA125 (Cancer Antigen 125); and CA19-9 (Carbohydrate Antigen 19-9).

6. The composition of claim 2, wherein the tumor antigen obtained from a cancer cell lysate, optionally, wherein the composition further comprises a cancer cell lysate.

7. (canceled)

8. The composition of claim 6, wherein the cancer cell is selected from the group consisting of Colorectal carcinoma, RCC (renal cell carcinoma), Prostate carcinoma, Cervical carcinoma, Melanoma, Lung carcinoma, breast carcinoma and B, T leukemia, lymphoma, pancreatic carcinoma, myeloma, nasopharyngeal carcinoma, and gastric cancer cells.

9. The composition of claim 1, further comprising an adjuvant.

10. The composition of claim 1, (a) prepared by indirectly loading the γδ-T cells with the one or more tumor antigens by contacting the γδ-T cells with a composition comprising TAA's, before EV isolation and/or (b) wherein the EVs are small EVs.

11. (canceled)

12. The composition of claim 10, comprising the Evs with a size of about 20-200 nm.

13. The composition of claim 10 comprising Evs with a size of about 100-200 nm.

14. The composition of claim 1, in an effective amount to: (a) promote dendritic cell maturation measured as upregulation of CD83 and MHC-II expression by at least 30%; or (b) in an effective amount that promote dendritic cell maturation measured as upregulation of CD83 and MHC-II expression by at least 50%.

15. (canceled)

16. A method of eliciting an immune response in a subject in need thereof, comprising administering the composition of claim 1, to the subject.

17. The method of claim 16, wherein the subject has been diagnosed with at least one type of cancer selected from the group consisting of Colorectal carcinoma, RCC (renal cell carcinoma), Prostate carcinoma, Cervical carcinoma, Melanoma, Lung carcinoma, breast carcinoma and B, T leukemia, lymphoma, pancreatic carcinoma, myeloma, nasopharyngeal carcinoma, and gastric cancer.

18. The method of claim 16, wherein the composition is administered intranasally or intravenously.

19. (canceled)

20. The method of claim 16, comprising tumor antigens obtained from a cancer cell lysate.

21. The method of claim 20, wherein the cancer cell is selected from the group consisting of Colorectal carcinoma, RCC (renal cell carcinoma), Prostate carcinoma, Cervical carcinoma, Melanoma, Lung carcinoma, breast carcinoma and B, T leukemia, lymphoma, pancreatic carcinoma, myeloma, nasopharyngeal carcinoma, and gastric cancer cells.

22. The method of claim 16, further comprising administering one or more chemotherapeutic agents to the subject: optionally, wherein the chemotherapeutic agent is selected from the group consisting of an alkylating agent, an antimetabolite, a topoisomerase inhibitor, a mitotic inhibitor and an antitumor antibiotic.

23. (canceled)

24. The method of claim 22, further comprising administering: (a) an alkylating agent, wherein the alkylating agent is selected from the group consisting of Altretamine; Bendamustine; Busulfan; Carboplatin; Chlorambucil; Cisplatin; Cyclophosphamide; Dacarbazine; Ifosfamide; Mechlorethamine; Melphalan; Oxaliplatin; Procarbazine; Temozolomide; Thiotepa; Trabectedin, Carmustine, Lomustine; Streptozocin; (b) an antimetabolite, selected 5-fluorouracil; 6-mercaptopurine; Azacitidine: Capecitabine; Cladribine; Clofarabine: Cytarabine; Decitabine; Floxuridine; Fludarabine; Gemcitabine; Hydroxyurea; Methotrexate; Nelarabine: Pemetrexed; Pentostatin; Pralatrexate; Thioguanine: Trifluridine/tipiracil combination; (c) a topoisomerase inhibitor, a mitotic inhibitor or an antitumor antibiotic wherein:
(i) the topoisomerase inhibitor is selected from the group consisting of Etoposide; Irinotecan; Irinotecan liposomal; Mitoxantrone, Teniposide and Topotecan;
(ii) the mitotic inhibitor is selected from the group consisting of Cabazitaxel, Docetaxel, Nab-paclitaxel, Paclitaxel, Vinblastine, Vincristine, Vinorelbine; and/or
(iii) the antitumor antibiotic is selected from the group consisting of Antitumor antibiotics include, Daunorubicin, Doxorubicin, Epirubicin, Idarubicin, Mitoxantrone, Valrubicin. Bleomycin, Dactinomycin, and Mitomycin-C.

25. (canceled)

26. (canceled)

27. (canceled)

28. (canceled)
